(19) United States
(12) Patent Application Publication
(10) Pub. No.: US 2026/0144769 A1
(43) Pub. Date: May 28, 2026

(54) 4-PHENYLBUTYRATE RESTORED GABA UPTAKE AND REDUCED SEIZURES IN SLC6A1 VARIANT-MEDIATED DISORDERS

(71) Applicant: Vanderbilt University, Nashville, TN (US)

(72) Inventor: Jing-Qiong KANG, Nashville, TN (US)

(21) Appl. No.: 19/121,616

(22) PCT Filed: Jun. 5, 2023
(86) PCT No.: PCT/US2023/024391

§ 371 Date: Apr. 16, 2025

Related U.S. Application Data
(60) Provisional application No. 63/380,613, filed on Oct. 24, 2022.

(51) Int. Cl. A61K 31/192; A61K 38/17; A61K 48/00; A61P 25/08

(52) U.S. Cl. CPC A61K 31/192; A61K 38/1787; A61K 48/005; A61P 25/08

(57) ABSTRACT

Described herein are methods for disease or disorder associated with GABA transporter 1 (GAT-1) dysfunction. In one aspect described herein, the disease or disorder is associated with one or more solute carrier Family 6 Member 1 (SLC6A1) mutations, and 4-phenylbutyrate, or the pharmaceutically acceptable salt thereof, is administered at a dose of 75 mg/kg/day to 125 mg/kg/day. In another embodiment, the method comprises combined GAT-1 gene therapy combined with administration of 4-phenylbutyrate.

### CROSS-REFERENCE TO RELATED APPLICATIONS

This application claims priority to U.S. Provisional Patent Application No. 63/380,613, filed on Oct. 24, 2022, the contents of which are incorporate by reference herein in its entirety.

### REFERENCE TO SEQUENCE LISTING

This application was filed with a Sequence Listing XML in ST.26 XML format accordance with 37 C.F.R. § 1.831 and PCT rule 13ter. The Sequence Listing XML file submitted in the USPTO Patent Center, “093386-9332-WO01_sequence_listing_xml_2-JUN-2023.xml,” was created on Jun. 2, 2023, contains 4 sequences, has a file size of 14.0 Kbytes, and is incorporated by reference in its entirety into the specification.

### FEDERALLY SPONSORED RESEARCH

This invention was made with government support under grant numbers NS082635 and NS121718 awarded by the National Institutes of Health. The government has certain rights in the invention.

### TECHNICAL FIELD

Described herein are methods for disease or disorder associated with GABA transporter 1 (GAT-1) dysfunction. In one aspect described herein, the disease or disorder is associated with one or more solute carrier Family 6 Member 1 (SLC6A1) mutations, and 4-phenylbutyrate, or the pharmaceutically acceptable salt thereof, is administered at a dose of 75 mg/kg/day to 125 mg/kg/day.

### BACKGROUND

Genetic variation and the subsequent protein misfolding are a major cause of disease throughout the life span of those affected by solute carrier family 6 member 1 disorders. In childhood, pathogenic variants, or mutations in numerous genes, give rise to a wide range of neurodevelopmental disorders. GABA transporter 1 (GAT-1) encoding SLC6A1 is one of such genes linked to potential pathogenic variants. SLC6A1 is a member of the neurotransmitter subgroup of the solute carrier (SLC6) family. The family includes 3 γ-aminobutyric acid (GABA) transporters (GAT), 2 glycine transporters (GLY), and the monoamine transporters such as dopamine (DAT), serotonin (SERT) and norepinephrine (NET). The SLC6 family transporters are integral membrane proteins characterized by the Na⁺-dependent translocation of small amino acid or amino acid-like substrates. Genetic sequencing has identified that mutations in the family are a major etiology for neurodevelopmental disorders such as epilepsy and autism.

The functional consequences of both missense and nonsense SLC6A1 mutations have been characterized and the common mechanisms of the molecular pathophysiology have been identified underlying the heterogeneous clinical phenotype. The molecular pathophysiological mechanisms include reduction or loss of GABA uptake, endoplasmic reticulum (ER) retention of the mutant GAT-1 protein, and reduced cell surface and total GAT-1 protein expression due to impaired protein trafficking. The mechanisms directly contributing to diminished GAT-1 function include decreased membrane protein trafficking due to protein misfolding and altered protein stability. Previous studies suggests that the mutant GAT-1 is subject to the common protein surveillance inside the ER as other mutations such as γ-aminobutyric acid type A (GABA_{A}) receptor subunit mutations.

Studies in the mutant GABA_{A} receptor and GAT-1 suggest that impaired protein trafficking due to protein misfolding is a common etiology in genetic epilepsy and neurodevelopmental disorders. This thus provides a great opportunity for treatment development by leveraging the protein trafficking pathway, via which the misfolded mutant protein is processed. Protein misfolding has been widely studied for later onset neurodegenerative diseases but much less so for early onset childhood disorders such as epilepsy. These findings on impaired protein trafficking in both GABA_{A} receptors and GAT-1 provide a mechanistic link between genetic epilepsy and neurodegenerative disease. This suggests pharmacological compounds identified for neurodegeneration could be repurposed for neurodevelopmental disorders such as genetic epilepsy. Targeting the ER pathway and promoting membrane protein trafficking could be a novel treatment target for genetic epilepsy as well as other related disorders.

4-phenylbutyrate (PBA), or 4-phenylbutyric acid, is a salt of an aromatic fatty acid. It is used to treat urea cycle disorders, as its metabolites offer an alternative pathway to the urea cycle, allowing excretion of excess nitrogen. PBA is a chaperone that can reduce ER stress while also functioning as a histone deacetylase inhibitor. In this study, the effect of PBA was compared with other chaperones on GABA uptake for eight mutations and evaluated the impact of PBA on the functional rescue in different model systems, including in patient induced pluripotent stem cell (iPSC) derived astrocytes and knockin mice. Because GAT-1 is expressed in both neurons and astrocytes and because of the unique role of GAT-1 in thalamic astrocytes and the associated seizures phenotypes, the impact of treatment on GABA uptake activity was characterized in astrocytes and neurons derived from patient cells and in two SLC6A1 mutation knockin mouse models. In knock-in mice, the effect of PBA by video monitoring synchronized EEG recordings was evaluated and the effect of PBA in mitigating seizure activity was determined.

### SUMMARY

One embodiment described herein is a method for treating a disease or disorder associated with GABA transporter 1 (GAT-1) dysfunction in a subject, the method comprising: administering to the subject a therapeutically effective amount of 4-phenylbutyrate, or a pharmaceutically acceptable salt thereof. In one aspect, the disease or disorder is associated with one or more solute carrier Family 6 Member 1 (SLC6A1) mutations. In another aspect, one or more of the SLC6A1 mutations is a missense or a nonsense mutation. In another aspect, one or more of the SLC6A1 mutations is a SLC6A1(A288V) mutation or a SLC6A1(S295L) mutation. In another aspect, the method increases GAT-1 function in a plurality of thalamic astrocytes in the subject. In another aspect, the method increases GAT-1 function in a plurality of neurons in the subject. In another aspect, the disease or disorder is a seizure disorder or a neurodevelopmental disorder. In another aspect, the disease or disorder is epilepsy, schizophrenia, autism spectrum disorder, attention-deficit/hyperactivity disorder (ADHD), or a learning disorder. In another aspect, the method reduces the occurrence of seizures. In another aspect, 4-phenylbutyrate, or the pharmaceutically acceptable salt thereof, is administered as a single daily dose. In another aspect, 4-phenylbutyrate, or the pharmaceutically acceptable salt thereof, is administered at a dose of 75-125 mg/kg/day.

Another embodiment described herein is a method for treating a disease or disorder associated with one or more solute carrier Family 6 Member 1 (SLC6A1) mutations in a subject, the method comprising: administering to the subject a therapeutically effective amount of 4-phenylbutyrate, or a pharmaceutically acceptable salt thereof. In one aspect, one or more of the SLC6A1 mutations is a missense or a nonsense mutation. In another aspect, one or more of the SLC6A1 mutations is a SLC6A1(A288V) mutation or a SLC6A1(S295L) mutation. In another aspect, the disease or disorder is a seizure disorder or a neurodevelopmental disorder. In another aspect, the disease or disorder is epilepsy, schizophrenia, autism spectrum disorder, attention-deficit/hyperactivity disorder (ADHD), or a learning disorder. In another aspect, the method reduces the occurrence of seizures. In another aspect, 4-phenylbutyrate, or the pharmaceutically acceptable salt thereof, is administered as a single daily dose. In another aspect, 4-phenylbutyrate, or the pharmaceutically acceptable salt thereof, is administered at 75-125 mg/kg/day.

Another embodiment described herein is a method for treating a disease or disorder associated with GABA transporter 1 (GAT-1) dysfunction in a subject in need thereof, the method comprising: administering to the subject a therapeutically effective amount of 4-phenylbutyrate, or a pharmaceutically acceptable salt thereof; and administering a therapeutically effective amount of GAT-1 gene therapy comprising a vector configured to augment wildtype GAT-1 expression in the subject in need thereof. In one aspect, the GAT-1 gene therapy comprises expression of wild type SLC6A1. In another aspect, the 4-phenylbutyrate and GAT-1 gene therapy function synergistically. In another aspect, the therapeutically effective amount of 4-phenylbutyrate is reduced as compared to mono therapy where 4-phenylbutyrate is administered alone.

Another embodiment described herein is the use of 4-phenylbutyrate, or a pharmaceutically acceptable salt thereof, for the preparation of a medicament for the treatment of a disease or disorder associated with GABA transporter 1 (GAT-1) dysfunction in a subject. In one aspect, the disease or disorder is a seizure disorder or a neurodevelopmental disorder. In another aspect, the disease or disorder is epilepsy, schizophrenia, autism spectrum disorder, attention-deficit/hyperactivity disorder (ADHD), or a learning disorder. In another aspect, the medicament reduces the occurrence of seizures.

Another embodiment described herein is the use of 4-phenylbutyrate, or a pharmaceutically acceptable salt thereof in combination with GAT-1 gene therapy for the preparation of a medicament for the treatment of a disease or disorder associated with GABA transporter 1 (GAT-1) dysfunction in a subject.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-D show reduced function and trafficking of the mutant GABA transporter 1 encoded by SLC6A1 variants associated with epilepsy, autism, ADHD, and intellectual delay. FIG. 1A shows a schematic presentation of mutant GABA transporter 1 (GAT-1) protein topology and locations of representative mutations in human SLC6A1 associated with various epilepsy syndromes and neurodevelopmental disorders. These mutations are distributed in various locations and domains of the encoded GAT-1 protein peptide. The large red dots represent the eight mutations evaluated in the study. FIG. 1B-D show HEK293T cells that were transfected with the wildtype or the mutant GAT-1^{YFP} for 48 hrs. FIG. 1B shows graphs representing the altered GABA reuptake function of the mutant GAT-1 encoded by 8 different SLC6A1 variants in HEK293T cells measured by the high-throughput ³H radio-labeling GABA uptake on a liquid scintillator with QuantaSmart. 966 stands for the wildtype treated with GAT-1 inhibitor CI-966 (50 μM) and NNC-711 for the wildtype treated with NNC-711 (35 μM) for 30 min before preincubation. FIGS. 1C-D show flow cytometry histograms depict the relative surface (FIG. 1C) or total (FIG. 1D) expression of the wildtype and the mutant GAT-1. The relative total expression level of GAT-1 in each mutant transporter was normalized to that obtained from cells with transfection of the wildtypes. (N=4-5 different transfections, δδδ P<0.001 overall mutations vs. wt, *P<0.05, **P<0.01, ***p<0.001 vs. wt, one-way analysis of variance (ANOVA) and Newman-Keuls test. Values were expressed as mean±S.E.M.)

FIGS. 2A-D show that all surveyed mutant GAT-1 transporters had less mature but more immature form of the GAT-1 protein. FIGS. 2A-B show the total lysates of HEK293T cells expressing the wildtype or variant GAT-1 were undigested (U) or digested with Endo-H (H) and then analyzed by SDS-PAGE. The membrane was immunoblotted with a rabbit anti-GAT-1. The red-boxed region represents the mature form of GAT-1 in cells. CHO stands for Chinese hamster ovary cells. CHO cells were used for control because of the low level of the endogenous GAT-1 expression. FIG. 2C shows a graph representing the normalized integrated protein density values (IDVs) of the mature form of GAT-1 defined by being Endo-H resistant normalized to the wildtype mature form of GAT-1 (bands 1+2). FIG. 2D. shows a graph representing the normalized integrated protein density values (IDVs) of the immature form of GAT-1 defined by being Endo-H unresistant (shifted to a lower level after H digestion) normalized to the wildtype immature form of GAT-1 (band 3 shifted to band 4). (N=4-5 different transfections, δδδ P<0.001 overall mutations vs. wt, *P<0.05, **P<0.01, ***p<0.001 vs. wt, one-way analysis of variance (ANOVA) and Newman-Keuls test. Values were expressed as mean±S.E.M.)

FIGS. 3A-B shows the effect of other chaperones like menthol on GABA uptake. HEK293T cells were transfected with wildtype (wt) or the mutant GAT-1YFP for 48 hr. FIG. 3A shows an experiment where menthol (125, 250, 500, or 1000 nM) or PBA (2 mM) was applied dropwise to each dish and incubated for 24 hrs. PBA from stocking solution (2 M) was diluted with DMEM 100 μL to desired concentration. The graph represents the altered GABA reuptake function of the wildtype GAT-1 in HEK293T cells treated with PBA for a series of different concentrations. The GABA uptake activity of cells treated with PBA of different concentrations was normalized to the sister cultures treated with DMSO alone for 24 hrs. The graph shows the GABA uptake function of the wildtype GAT-1, or the mutant GAT-1(A288V) and GAT-1(S295L) treated with menthol or PBA. FIG. 3B shows the GABA uptake function of the wildtype GAT-1 or the mutant GAT-1 treated with menthol (1000 nM). (*p<0.05; ***p<0.001 vs DMSO, n=4-5 transfections).

FIG. 4A-D show 4-Phenylbutyric acid (PBA) concentration and time-dependently increased the GABA uptake in cells expressing the wildtype or the mutant GAT-1. FIG. 4A-B show HEK293T cells that were transfected with wildtype GAT-1^{YFP} (wt) for 48 hr., PBA (2 mM) was applied dropwise to each dish at different concentrations (FIG. 4A) and incubated for time durations (FIG. 4B). PBA from stocking solution (2 M) was diluted with DMEM 100 μL to desired concentration. FIG. 4A represent the altered GABA reuptake function of the wildtype GAT-1^{YFP} in HEK293T cells treated with PBA for a series of different concentrations. The GABA uptake activity of cells treated with PBA of different concentrations was normalized to the sister cultures treated with DMSO alone for 24 hrs. FIG. 4B represent the altered GABA reuptake function of the wildtype GAT-1 in HEK293T cells treated with PBA for a series of different time duration over treated with DMSO alone for 24 hrs. GABA uptake activity was measured by the high-throughput 3H radio-labeling GABA uptake on a liquid scintillator with QuantaSmart. FIG. 4C shows a cartoon illustrating that only mutant allele was expressed. HEK293T cells were transfected with the wildtype or the mutant GAT-1^{YFP} cDNAs alone for 48 hr. FIG. 4C shows a cartoon illustrating the coexistence condition of the wildtype and the mutant allele in patients and both the wildtype and the mutant alleles were expressed. HEK293T cells were transfected with the wildtype GAT-1^{YFP} alone or in mixture of the wildtype or the mutant cDNAs for 48 hr. In the mixed condition, the ratio of the wildtype GAT-1 with pcDNA or the mutant cDNAs are 1:1 with the total cDNA amount of 0.5 μg. PBA (2 mM) was applied for 24 hrs while DMSO was applied as control. Both wt and the mutant were normalized to its own DMSO treated conditions. 966 stands for the wildtype treated with CI-966 (50 μM) while 711 stands for NNC-711 (35 μM). (N=4-7 different transfections. In FIG. 4A and FIG. 4B, *P<0.05, **P<0.01, ***p<0.001 vs. wt 0. In FIG. C and FIG. 4D, *P<0.05, **P<0.01, ***p<0.001 vs. its own DMSO treated. In FIG. 4C, ns stands for no significance. One-way analysis of variance (ANOVA) and Newman-Keuls test. Values were expressed as mean±S.E.M.

FIGS. 5A-F show 4-Phenylbutyrate (PBA) rescued the GABA uptake function in the patient astrocytes and neurons. FIG. 5A shows human astrocytes and inhibitory neurons were differentiated from the neural progenitor cells (NPCs) from the patient and the CRISPR corrected isogenic control line. Live images of human neural progenitor cells (NPCs), astrocytes and GABAergic inhibitory neurons differentiated from the human induced pluripotent stem cells (iPSCs) on the day of experiment. FIGS. 5B-E show astrocytes at day 30-35 (FIGS. 5B-C) or neurons at day 60-65 (FIGS. 5D-E) after differentiation were treated with PBA (2 mM) for 24 hrs before 3H radioactive GABA uptake assay. The DMSO treated corrected or patient cells was taken as 1. FIG. 5F shows corrected astrocytes at day 30-35 after differentiation were transfected with the wildtype or the mutant GAT-1^{YFP} cDNAs (1 μg per a 35 mm² dish) for 48 hrs before 3H radioactive GABA uptake assay. PBA (2 mM) was applied for 24 hrs before GABA uptake assay experiment. GABA flux was measured after 30 min transport at room temperature. The influx of GABA, expressed in pmol/μg protein/min, was averaged from duplicates for each condition and for each transfection. The average counting was DMSO treated condition taken as 1. In FIG. 5B, FIG. 5D and FIG. 5F, 966 stands for CI-966 (50 μM) and 711 stands for NNC-711 (35 μM) that was applied 30 min to the astrocytes transfected with the wildtype GAT-1^{YFP} before preincubation and removed during preincubation. In FIGS. 5B and D, ***p<0.001 vs. corrected DMSO treated. § § <0.01, § § § p<0.001 vs. patient DMSO treated. In FIG. 5C and FIG. 5E, the PBA treated corrected or patient GABA uptake was normalized to its DMSO treated. In FIG. 5C, FIG. 5E and FIG. 5F, the PBA treated corrected/wildtype or patient/mutant GABA uptake was normalized to its DMSO treated. *p<0.05; **p<0.01; ***p<0.001 vs. DMSO treated in its own group. In FIGS. 5B-E, n=4-8 batches of cells. In FIG. 5F, n=4-5 different transfections). Unpaired t test was used for FIG. 5C and FIG. 5E. One-way analysis of variance followed by a Dunnett post hoc multiple comparison test was used in FIG. 5B, FIG. 5D and FIG. 5F. Values were expressed as mean±S.E.M.).

FIGS. 6A-E show patient astrocytes caused ER retention of the wildtype and exacerbated the mutant GAT-1 ER retention while 4-Phenylbutyrate (PBA) increased GAT-1 protein expression. FIGS. 6A-B show human patient corrected (isogenic control, Corr) or uncorrected patient (Pat) astrocytes at day 30-35 after differentiation from iPSCs were co-transfected with the endoplasmic reticulum (ER) marker ER^{CFP} in combination with the wildtype or the mutant GAT-1^{YFP} cDNAs (0.5 μg: 0.5 μg per a 35 mm² dish) for 48 hrs before confocal microscopy analysis. Confocal images were acquired in live astrocytes under 63× objective with zoom under 2.5×. FIG. 6A shows purple boxed regions were enlarged. FIG. 6B represents the ER overlapping signal of GAT-1^{YFP} analyzed by Metamorph. FIG. 6C shows total cell lysates from astrocytes cultured in 100 mm² dishes treated with DMSO or PBA (2 mM) for 24 hrs and were analyzed with SDS-PAGE. Membranes were blotted with a rabbit polyclonal anti-GAT-1 antibody (FIG. 6C). The lysates of Chinese hamster ovary (CHO) cells were used as control. FIGS. 6D-E show the protein IDVs of the corrected or patient GAT-1 and in human astrocytes was normalized to the corrected astrocytes treated with DMSO, the GAT-1 protein was normalized to its own internal control ATPase or GAPDH and then to the DMSO treated corrected levels, which is arbitrarily taken as 1 (FIG. 6D) or its own genotype but DMSO treated, which is taken as 1 (FIG. 6E). Values were expressed as mean±S.E.M.). In FIG. 6B, N=8-11 culture replicates. In FIGS. 6C-E, N=6 batch of cells. In FIG. 6B, ***p<0.001 vs. corrected. § § § P<0.001 vs. wt in patient cells. In FIG. 6D, ***P<0.001 corrected untreated. § § § P<0.001 vs. Patient untreated. In FIG. 6E, ***p<0.001 vs. its own untreated; § § § P<0.001 vs. corrected PBA treated, two-way analysis of variance followed by Bonferroni multiple comparison test).

FIGS. 7A-D show 4-Phenylbutyrate (PBA) rescued the GABA uptake in cortical astrocytes and neurons in SLC6A1^{+/A288V} and SLC6A1^{+/S295L} mice. Mouse cortical astrocytes or neurons were cultured from postnatal pus at day 0-3 days old for astrocytes and day 0 for neurons from the SLC6A1^{+/A288V} or SLC6A1^{+/S295L} mouse line. FIGS. 7A-B show astrocytes under passage 2 were grown in 100-mm² dishes and passaged into 35-mm² dishes before GABA uptake assay.

FIGS. 7C-D show neurons directly cultured in the 35-mm² dishes and GABA uptake was evaluated between day 15-17 days after culture. CI-966 (50 μM) and NNC711 (35 μM) was applied 30 min before preincubation and removed during preincubation. GAT-3 inhibitor SNAP5114 (30 μM) was applied during GABA uptake to make sure only GAT-1 activity was measured. The wildtype astrocytes (FIGS. 7A-B) or cortical neurons (FIGS. 7C-D) of either SLC6A1^{+/A288V} or SLC6A1^{+/S295L} were taken as 1. The cultures derived from each mutant mouse was compared to the culture from its own wildtype littermates. In FIG. 7B and FIG. 7D, sister cultures of astrocytes or neurons from different mouse lines were incubated with DMSO or PBA 2 mM for 24 hr. before GABA uptake. The wildtype data were pooled from two mouse lines. The GABA uptake activity of PBA treated was normalized to its own DMSO conditions. The graph represents the relative GABA uptake level normalized to cells of its own genotype treated with DMSO. CI-966 (50 μM) or NNC-711 (35 μM) was applied 30 min before preincubation and removed during preincubation. Two-way analysis of variance followed by Bonferroni multiple comparison test was used. Values were expressed as mean±S.E.M. In FIG. 7A and FIG. 7C, ***p<0.001 vs. wt; § § § P<0.001 S295L vs. A288V. In FIG. 7B and FIG. 7D, ***p<0.001 vs. untreated; § § P<0.01 S295L vs. wt treated; δδP<0.01 vs. A288V treated. N=4-9 batches of astrocytes from 4 pairs of littermates for FIG. 7A. N=5-8 batches of astrocytes from 4 pairs of littermates for FIG. 7B; N=4-7 batches of neuron cultures from 5 litters of A288V and 6 litters of S295L) in FIG. 7C and FIG. 7D.

FIGS. 8A-H show both SLC6A1^{+/A288V} and SLC6A1^{+/S295L} mice had reduced GAT-1 protein that was partially restored by 4-Phenylbutyrate (PBA). FIGS. 8A-H show lysates from different brain regions (cortex (cor), cerebellum (cb), hippocampus (hip) and thalamus (thal)) from the wildtype (wt) and heterozygous (het) mice at 4-6 months old, untreated (FIGS. 8A-B) or treated with vehicle or PBA (100 mg/kg) for 7 days (FIG. 8E-F) were subjected to SDS-PAGE and immunoblotted with anti-GAT-1 antibody. FIGS. 8C-D show integrated density values (IDVs) for total GAT-1 from wild-type and het KI were normalized to the Na⁺/K⁺ ATPase or anti-glyceraldehyde-3-phosphate dehydrogenase (GAPDH) loading control (LC) in each specific brain region and plotted. N=4 from 4 pairs of mice. FIG. 8G-H show integrated density values (IDVs) for total GAT-1 from het KI treated with vehicle or treated with PBA were normalized to the Na⁺/K⁺ ATPase or anti-glyceraldehyde-3-phosphate dehydrogenase (GAPDH) loading control (LC). The IDVs of the heterozygous treated with PBA were then normalized to vehicle treated. The vehicle treated in each brain region was taken as 1. N=4 from 4 pairs of mice for FIG. 8C, FIG. 8D, FIG. 8G and FIG. 8H, Values were expressed as mean±S.E.M. One way ANOVA or unpaired t test. In FIG. 8C and FIG. 8D, ***p<0.001 vs. wt, in FIG. 8G and FIG. 8H, **p<0.01; ***p<0.001 vs. vehicle treated).

FIGS. 9A-C show the GABA_{A} receptor γ2 subunit protein was not increased in 4-phenylbutyrate treated Slc6a1^{+/S295L} mice. FIGS. 9A-B show full-length gels for the GABA_{A} receptor γ2 subunit protein in different brain regions of the heterozygous mice treated with vehicle or PBA. FIG. 9C shows a graph illustrating the fold change of protein IDVs from mice treated with PBA normalized to its loading control ATPase and then to the vehicle treated. The heterozygous (het) mice at 2-8 months old were treated with vehicle or PBA (100 mg/kg) for 7 days. The total lysates from cortex (cor), cerebellum (cb), hippocampus (hip) and thalamus (thal) were subjected to SDS-PAGE and immunoblotted with rabbit anti-γ2 antibody. Integrated density values (IDVs) of the γ2 subunit from het mice treated with PBA were normalized to the Na⁺/K⁺ ATPase or anti-glyceraldehyde-3-phosphate dehydrogenase (GAPDH) loading control (LC) and then to the vehicle treated, which is arbitrarily taken as 1 in each specific brain region. N=4 blots from 4 pairs of mice.

FIGS. 10A-G show 4-Phenylbutyrate (PBA) alone reduced seizures in mutation knockin mice. FIG. 10A shows a schematic depiction of experimental paradigm for EEG recordings and PBA treatment. FIG. 10B shows representative EEG recordings demonstrating that the heterozygous SLC6A1^{+/S295L} (het) KI mice had frequent absence like spike wave discharges (SWDs) and some myoclonic jerks during baseline recordings. FIG. 10C shows a comparison of EEG traces recorded after the vehicle (normal saline 100 μl) treated or after treatment with PBA (100 mg/kg, ip, single dose, daily) for 7 days. FIG. 10D shows a graph showing the total number of 5-7 Hz SWDs calculated by Seizure Pro during 48 hrs recordings after vehicle or PBA treatment. PBA treatment (100 mg/kg, ip, single dose, daily) for 7 days reduced seizure activity. **p<0.01; vs. vehicle treated, paired t test, N=6 animals. Values were expressed as mean±S.E.M. FIG. 10E-F show graphs illustrating the percentage of seizure remaining (FIG. 10E) and seizure reduction (FIG. 10F) in each mouse after PBA treatment (N=6 animals, paired t test). FIG. 10G shows dual therapy as a feasible approach for treating SLC6A1 variants and other genetic disorders by boosting the wildtype allele, removing the mutant allele and ER stress with PBA.

FIG. 11 shows a synopsis of each individual therapy and the combined gene therapy in combination with PBA.

FIG. 12 shows GABA uptake function was assessed in HEK cells transfected with increasing doses of GAT-1 cDNA. 0.25 μg of cDNA was used as a baseline, relative to which the fold GABA uptake from each other treatment group was compared. There were significant (P<0.05) differences in GABA uptake between 0.25 μg and 2 μg, 0.50 μg and 2 μg, and 1 μg vs 2 μg.

FIG. 13 shows GABA uptake function was assessed in HEK cells transfected with either 0.5 μg of GAT-1 cDNA alone or 0.25 μg of GAT-1 cDNA in combination with 2 mM PBA treatment. No significant difference in the amount of GABA uptake was observed between the cDNA alone treatment and the combined cDNA/PBA treatment.

FIGS. 14A-B show RT-qPCR data from a cohort of nine wild-type mice (FIG. 14A) and seven SLC6A1^{+/S295L} mice (FIG. 14B). Five of the wild-type mice and three of the SLC6A1^{+/S295L} mice were treated with PBA for 7 days prior to tissue collection, while the remainder (four wild-type, four SLC6A1^{+/S295L}) were left untreated. There was no significant difference in the amount of SLC6A1 gene expression detected between the treated and untreated mice. Total RNA was extracted from mouse whole-brain tissue.

FIG. 15 shows a comparison between GABA uptake function in HEK cells transfected with cDNA alone versus HEK cells transfected with an equal amount of cDNA in conjunction with PBA treatment.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a mammal. In another embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

One embodiment described herein is a method for treating a disease or disorder associated with GABA transporter 1 (GAT-1) dysfunction in a subject, the method comprising: administering to the subject a therapeutically effective amount of 4-phenylbutyrate, or a pharmaceutically acceptable salt thereof. In one aspect, the disease or disorder is associated with one or more solute carrier Family 6 Member 1 (SLC6A1) mutations. In another aspect, one or more of the SLC6A1 mutations is a missense or a nonsense mutation. In another aspect, one or more of the SLC6A1 mutations is a SLC6A1(A288V) mutation or a SLC6A1(S295L) mutation. In another aspect, the method increases GAT-1 function in a plurality of thalamic astrocytes in the subject. In another aspect, the method increases GAT-1 function in a plurality of neurons in the subject. In another aspect, the disease or disorder is a seizure disorder or a neurodevelopmental disorder. In another aspect, the disease or disorder is epilepsy, schizophrenia, autism spectrum disorder, attention-deficit/hyperactivity disorder (ADHD), or a learning disorder. In another aspect, the method reduces the occurrence of seizures. In another aspect, 4-phenylbutyrate, or the pharmaceutically acceptable salt thereof, is administered as a single daily dose. In another aspect, 4-phenylbutyrate, or the pharmaceutically acceptable salt thereof, is administered at a dose of 75-125 mg/kg/day.

Another embodiment described herein is a method for treating a disease or disorder associated with one or more solute carrier Family 6 Member 1 (SLC6A1) mutations in a subject, the method comprising: administering to the subject a therapeutically effective amount of 4-phenylbutyrate, or a pharmaceutically acceptable salt thereof. In one aspect, one or more of the SLC6A1 mutations is a missense or a nonsense mutation. In another aspect, one or more of the SLC6A1 mutations is a SLC6A1(A288V) mutation or a SLC6A1(S295L) mutation. In another aspect, the disease or disorder is a seizure disorder or a neurodevelopmental disorder. In another aspect, the disease or disorder is epilepsy, schizophrenia, autism spectrum disorder, attention-deficit/hyperactivity disorder (ADHD), or a learning disorder. In another aspect, the method reduces the occurrence of seizures. In another aspect, 4-phenylbutyrate, or the pharmaceutically acceptable salt thereof, is administered as a single daily dose. In another aspect, 4-phenylbutyrate, or the pharmaceutically acceptable salt thereof, is administered at 75-125 mg/kg/day.

Another embodiment described herein is a method for treating a disease or disorder associated with GABA transporter 1 (GAT-1) dysfunction in a subject in need thereof, the method comprising: administering to the subject a therapeutically effective amount of 4-phenylbutyrate, or a pharmaceutically acceptable salt thereof; and administering a therapeutically effective amount of GAT-1 gene therapy comprising a vector configured to augment wildtype GAT-1 expression in the subject in need thereof. In one aspect, the GAT-1 gene therapy comprises expression of wild type SLC6A1. In another aspect, the 4-phenylbutyrate and GAT-1 gene therapy function synergistically. In another aspect, the therapeutically effective amount of 4-phenylbutyrate is reduced as compared to mono therapy where 4-phenylbutyrate is administered alone.

Another embodiment described herein is the use of 4-phenylbutyrate, or a pharmaceutically acceptable salt thereof, for the preparation of a medicament for the treatment of a disease or disorder associated with GABA transporter 1 (GAT-1) dysfunction in a subject. In one aspect, the disease or disorder is a seizure disorder or a neurodevelopmental disorder. In another aspect, the disease or disorder is epilepsy, schizophrenia, autism spectrum disorder, attention-deficit/hyperactivity disorder (ADHD), or a learning disorder. In another aspect, the medicament reduces the occurrence of seizures.

Another embodiment described herein is the use of 4-phenylbutyrate, or a pharmaceutically acceptable salt thereof in combination with GAT-1 gene therapy for the preparation of a medicament for the treatment of a disease or disorder associated with GABA transporter 1 (GAT-1) dysfunction in a subject.

### Pharmaceutically Acceptable Salts

Disclosed compounds (e.g., 4-phenylbutyrate or 4-phenylbutyric acid) may exist as pharmaceutically acceptable salts. The term “pharmaceutically acceptable salt” refers to salts or zwitterions of the compounds which are water or oil-soluble or dispersible, suitable for treatment of disorders without undue toxicity, irritation, and allergic response, commensurate with a reasonable benefit/risk ratio and effective for their intended use. The salts may be prepared during the final isolation and purification of the compounds or separately by reacting the compound with a suitable acid. For example, a compound may be dissolved in a suitable solvent, such as but not limited to methanol and water and treated with at least one equivalent of an acid, like hydrochloric acid. The resulting salt may precipitate out and be isolated by filtration and dried under reduced pressure. Alternatively, the solvent and excess acid may be removed under reduced pressure to provide a salt. Representative salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, isethionate, fumarate, lactate, maleate, methanesulfonate, naphthylenesulfonate, nicotinate, oxalate, pamoate, pectinate, persulfate, 3-phenylpropionate, picrate, oxalate, maleate, pivalate, propionate, succinate, tartrate, thrichloroacetate, trifluoroacetate, glutamate, para-toluenesulfonate, undecanoate, hydrochloric, hydrobromic, sulfuric, phosphoric and the like. The amino groups of the compounds may also be quaternized with alkyl chlorides, bromides, and iodides such as methyl, ethyl, propyl, isopropyl, butyl, lauryl, myristyl, stearyl and the like.

Basic addition salts may be prepared during the final isolation and purification of the disclosed compounds by reaction of the carboxyl group with a suitable base such as the hydroxide, carbonate, or bicarbonate of a metal cation such as lithium, sodium, potassium, calcium, magnesium, or aluminum, or an organic primary, secondary, or tertiary amine. Quaternary amine salts can be prepared, such as those derived from methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine and N,N′-dibenzylethylenediamine, ethylenediamine, ethanolamine, diethanolamine, piperidine, piperazine, and the like.

### Pharmaceutical Compositions

4-Phenylbutryrate or a pharmaceutically acceptable salt thereof may present in a pharmaceutical composition comprising 4-phenylbutryrate or a pharmaceutically acceptable salt thereof, and a pharmaceutically acceptable carrier, diluent, or excipient. In exemplary aspects, the pharmaceutical compositions comprise a pharmaceutically acceptable carrier. As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the U.S. Pharmacopeia for use in animals, including humans.

The pharmaceutical composition in various aspects may comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, 18^{th} Edition, E. W. Martin (Mack Publishing Co., Easton, Pa., 1980), which is incorporated by reference in its entirety.

In some aspects, the pharmaceutical composition comprises formulation materials that are nontoxic to recipients at the dosages and concentrations employed. In specific embodiments, pharmaceutical compositions comprising 4-phenylbutryrate or a pharmaceutically acceptable salt thereof, and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In exemplary aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In some instances, the pharmaceutical composition may comprise formulation materials for modifying, maintaining, or preserving, for example, the pH, osmolarity, viscosity, clarity, color, isotonicity, odor, sterility, stability, rate of dissolution or release, adsorption, or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; syrup and other carbohydrates (such as glucose, mannose or dextrins); sugar-free syrup; proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapol); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, Remington's Pharmaceutical Sciences, 18^{th} Edition, (A. R. Genrmo, ed.), 1990, Mack Publishing Company.

The pharmaceutical compositions in various instances are formulated to achieve a physiologically compatible pH. In exemplary embodiments, the pH of the pharmaceutical composition is for example between about 4 or about 5 and about 8.0 or about 4.5 and about 7.5 or about 5.0 to about 7.5. In exemplary embodiments, the pH of the pharmaceutical composition is between 5.5 and 7.5.

The pharmaceutical composition may be administered to a subject via parenteral, nasal, oral, pulmonary, topical, vaginal, rectal, or cerebrospinal fluid (CSF) administration. For example, parenteral administration includes intrathecal, intracerebroventricular, intraparenchymal, intravenous, and a combination thereof. The following discussion on routes of administration is merely provided to illustrate exemplary embodiments and should not be construed as limiting the scope in any way.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The term, “parenteral” means not through the alimentary canal but by some other route such as subcutaneous, intramuscular, intraspinal, or intravenous.

4-Phenylbutryrate or a pharmaceutically acceptable salt thereof may be administered with a physiologically acceptable diluent in a pharmaceutical carrier, such as a sterile liquid or mixture of liquids, including, without limitation, water, saline, aqueous dextrose, and related sugar solutions, syrup including sugar-free syrup, an alcohol, such as ethanol or hexadecyl alcohol, a glycol, such as propylene glycol or polyethylene glycol, dimethylsulfoxide, glycerol, ketals such as 2,2-dimethyl-153-dioxolane-4-methanol, ethers, poly(ethyleneglycol) 400, oils, fatty acids, fatty acid esters or glycerides, or acetylated fatty acid glycerides with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include, without limitation, petroleum, animal, vegetable, or synthetic oils. Specific examples of oils include peanut, soybean, sesame, cottonseed, corn, olive, petrolatum, and mineral. Suitable fatty acids for use in parenteral formulations include, without limitation, oleic acid, stearic acid, and isostearic acid. Ethyl oleate and isopropyl myristate are examples of suitable fatty acid esters.

Suitable soaps for use in parenteral formulations include, without limitation, fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-β-aminopropionates, and 2-alkyl-imidazoline quaternary ammonium salts, and (e) mixtures thereof.

The parenteral formulations in some embodiments may contain 4-phenylbutryrate or a pharmaceutically acceptable salt thereof in solution. Preservatives and buffers can be used. In order to minimize or eliminate irritation at the site of injection, such compositions can contain one or more nonionic surfactants having a hydrophile-lipophile balance (HLB) of from about 12 to about 17. Suitable surfactants include, without limitation, polyethylene glycol sorbitan fatty acid esters, such as sorbitan monooleate and the high molecular weight adducts of ethylene oxide with a hydrophobic base, formed by the condensation of propylene oxide with propylene glycol. The parenteral formulations may be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions may be prepared from sterile powders, granules, and tablets of the kind previously described.

4-Phenylbutryrate or a pharmaceutically acceptable salt thereof may be present in an injectable formulation. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Toissel, 4th ed., 622-630 (1986)).

Formulations suitable for oral administration in some aspects comprise (a) liquid solutions, such as an effective amount of 4-phenylbutryrate or a pharmaceutically acceptable salt thereof dissolved in diluents, such as water, saline, syrups or orange juice; (b) capsules, sachets, tablets, lozenges, and troches, each containing a predetermined amount of 4-phenylbutryrate or a pharmaceutically acceptable salt thereof, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant. Capsule forms can be of the ordinary hard- or soft-shelled gelatin type containing, for example, surfactants, lubricants, and inert fillers, such as lactose, sucrose, calcium phosphate, and corn starch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and other pharmacologically compatible excipients.

### Dosings

4-Phenylbutyrate, or a pharmaceutically acceptable salt thereof, may be administered as a single daily dose. 4-Phenylbutyrate, or a pharmaceutically acceptable salt thereof, may be administered at a dose of 75-125 mg/kg/day. In various instances, 4-phenylbutyrate, or a pharmaceutically acceptable salt thereof may be administered at a dose of 80 mg/kg/day to 120 mg/kg/day; 85 mg/kg/day to 115 mg/kg/day: 90 mg/kg/day to 110 mg/kg/day; or 95 mg/kg/day to 105 mg/kg/day. In various instances, 4-phenylbutyrate, or a pharmaceutically acceptable salt thereof may be administered at a dose of no greater than 125 mg/kg/day; no greater than 120 mg/kg/day; no greater than 115 mg/kg/day; no greater than 110 mg/kg/day; no greater than 105 mg/kg/day; no greater than 100 mg/kg/day; no greater than 95 mg/kg/day; no greater than 90 mg/kg/day; no greater than 85 mg/kg/day; no greater than 80 mg/kg/day; or no greater than 75 mg/kg/day. In various instances, or a pharmaceutically acceptable salt thereof may be administered at a dose of no less than 75 mg/kg/day; no less than 80 mg/kg/day; no less than 85 mg/kg/day; no less than 90 mg/kg/day; no less than 95 mg/kg/day; no less than 100 mg/kg/day; no less than 105 mg/kg/day; no less than 110 mg/kg/day; no less than 115 mg/kg/day; or no less than 120 mg/kg/day.

### Combination Therapy

Another embodiment described herein is a method for treating a disease or disorder associated with GABA transporter 1 (GAT-1) dysfunction in a subject in need thereof, the method comprising: administering to the subject a therapeutically effective amount of 4-phenylbutyrate, or a pharmaceutically acceptable salt thereof; and administering a therapeutically effective amount of GAT-1 gene therapy comprising a vector configured to augment wildtype GAT-1 expression in the subject in need thereof. In one aspect, the GAT-1 gene therapy comprises expression of wild type SLC6A1. In another aspect, the 4-phenylbutyrate and GAT-1 gene therapy function synergistically. In another aspect, the therapeutically effective amount of 4-phenylbutyrate is reduced as compared to mono therapy where 4-phenylbutyrate is administered alone.

### Diseases and Disorders

The disease or disorder may be associated with GABA transporter 1 (GAT-1) dysfunction. In various instances, the disease or disorder may be associated with one or more solute carrier Family 6 Member 1 (SLC6A1) mutations. In some instances, one or more of the SLC6A1 mutations is a SLC6A1(A288V) mutation or a SLC6A1(S295L) mutation. The disease or disorder may be a seizure disorder or a neurodevelopmental disorder.

As used herein, the term “seizure disorder” is a medical condition characterized by episodes of uncontrolled electrical activity in the brain, thus producing symptoms that include two or more seizures. In various aspects, the seizure disorder is epilepsy (i.e., epileptic seizure disorder), simple partial seizure, benign rolandic epilepsy, catamenial epilepsy, atonic seizure, absence seizure, clonic seizure, tonic seizure, febrile seizure. In various aspects, the subject suffers from focal seizures, temporal lobe seizures, frontal lobe seizures, occipital lobe seizures, parietal lobe seizures, generalized seizures, absence seizures, myoclonic seizures, generalized convulsive seizures, generalized tonic-clonic seizures, symptomatic generalized epilepsy, progressive myoclonic epilepsy, or reflex epilepsy.

As used herein, the term “neurodevelopmental disorder” is a group of diseases arising due to a flaw in a developmental period (such as the fetal period, infancy, childhood, or puberty) of the nervous system. Examples of neurodevelopmental disorders, include, without limitation, autism spectrum disorder, attention-deficit/hyperactivity disorder (ADHD), and learning disorders.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described.

The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for treating a disease or disorder associated with GABA transporter 1 (GAT-1) dysfunction in a subject, the method comprising:
   administering to the subject a therapeutically effective amount of 4-phenylbutyrate, or a pharmaceutically acceptable salt thereof.
Clause 2. The method of clause 1, wherein the disease or disorder is associated with one or more solute carrier Family 6 Member 1 (SLC6A1) mutations.
Clause 3. The method of clause 2, wherein one or more of the SLC6A1 mutations is a missense or a nonsense mutation.
Clause 4. The method of any one of clauses 1-3, wherein one or more of the SLC6A1 mutations is a SLC6A1(A288V) mutation or a SLC6A1(S295L) mutation.
Clause 5. The method of any one of clauses 1-4, wherein the method increases GAT-1 function in a plurality of thalamic astrocytes in the subject.
Clause 6. The method of any one of clauses 1-5, wherein the method increases GAT-1 function in a plurality of neurons in the subject.
Clause 7. The method of any one of clauses 1-6, wherein the disease or disorder is a seizure disorder or a neurodevelopmental disorder.
Clause 8. The method of any one of clauses 1-7, wherein the disease or disorder is epilepsy, schizophrenia, autism spectrum disorder, attention-deficit/hyperactivity disorder (ADHD), or a learning disorder.
Clause 9. The method any one of clauses 1-8, wherein the method reduces the occurrence of seizures.
Clause 10. The method of any one of clauses 1-9, wherein 4-phenylbutyrate, or the pharmaceutically acceptable salt thereof, is administered as a single daily dose.
Clause 11. The method of any one of clauses 1-10, wherein 4-phenylbutyrate, or the pharmaceutically acceptable salt thereof, is administered at a dose of 75 mg/kg/day to 125 mg/kg/day.
Clause 12. A method for treating a disease or disorder associated with one or more solute carrier Family 6 Member 1 (SLC6A1) mutations in a subject, the method comprising:
   administering to the subject a therapeutically effective amount of 4-phenylbutyrate, or a pharmaceutically acceptable salt thereof.
Clause 13. The method of clause 12, wherein one or more of the SLC6A1 mutations is a missense or a nonsense mutation.
Clause 14. The method of clause 12 or 13, wherein one or more of the SLC6A1 mutations is a SLC6A1(A288V) mutation or a SLC6A1(S295L) mutation.
Clause 15. The method of any one of clauses 12-14, wherein the disease or disorder is a seizure disorder or a neurodevelopmental disorder.
Clause 16. The method of any one of clauses 12-15, wherein the disease or disorder is epilepsy, schizophrenia, autism spectrum disorder, attention-deficit/hyperactivity disorder (ADHD), or a learning disorder.
Clause 17. The method of any one of clauses 12-16, wherein the method reduces the occurrence of seizures.
Clause 18. The method of any one of clauses 12-17, wherein 4-phenylbutyrate, or the pharmaceutically acceptable salt thereof, is administered as a single daily dose.
Clause 19. The method of any one of clauses 12-18, wherein 4-phenylbutyrate, or the pharmaceutically acceptable salt thereof, is administered at 75-125 mg/kg/day.
Clause 20. A method for treating a disease or disorder associated with GABA transporter 1 (GAT-1) dysfunction in a subject in need thereof, the method comprising:
   administering to the subject a therapeutically effective amount of 4-phenylbutyrate, or a pharmaceutically acceptable salt thereof; and
   administering a therapeutically effective amount of GAT-1 gene therapy comprising a vector configured to augment wildtype GAT-1 expression in the subject in need thereof.
Clause 21. The method of clause 20, wherein the GAT-1 gene therapy comprises expression of wild type SLC6A1.
Clause 22. The method of clause 20 or 21, wherein the 4-phenylbutyrate and GAT-1 gene therapy function synergistically.
Clause 23. The method of any one of clauses 20-22, wherein the therapeutically effective amount of 4-phenylbutyrate is reduced as compared to mono therapy where 4-phenylbutyrate is administered alone.
Clause 24. The use of 4-phenylbutyrate, or a pharmaceutically acceptable salt thereof, for the preparation of a medicament for the treatment of a disease or disorder associated with GABA transporter 1 (GAT-1) dysfunction in a subject.
Clause 25. The use of clause 24, wherein the disease or disorder is a seizure disorder or a neurodevelopmental disorder.
Clause 26. The use of clause 24 or 25, wherein the disease or disorder is epilepsy, schizophrenia, autism spectrum disorder, attention-deficit/hyperactivity disorder (ADHD), or a learning disorder.
Clause 27. The use of any one of clauses 24-26, wherein the medicament reduces the occurrence of seizures.
Clause 28. The use of 4-phenylbutyrate, or a pharmaceutically acceptable salt thereof in combination with GAT-1 gene therapy for the preparation of a medicament for the treatment of a disease or disorder associated with GABA transporter 1 (GAT-1) dysfunction in a subject.

### EXAMPLES

### Example 1

### SLC6A1 Variant Information

The patient variants are selected from the lab cDNA library built based on previous studies. Those variants represent both missense and nonsense mutations, exhibit variant locations in both the N terminus and C-terminus, and display common patterns of functioning and trafficking defects of the variants.

The human wild-type solute carrier family 6-member 1 (SLC6A1) mRNA transcript and encoded protein are available in Genbank at Accession No. NM_001348250.2 and NP_001335179.1, and SEQ ID NO: 1-2, respectively. The mouse wild-type solute carrier family 6-member 1 (SLC6A1) mRNA transcript and encoded protein are available in Genbank at Accession No. NM_178703.5 and NP_848818.1, and SEQ ID NO: 3-4, respectively. The protein sequences for human and mouse SLC6A1 are provided in Table 1.

TABLE 1
Human and Mouse SLC6A1 Protein Sequences

| | |
|---|---|
| Polypeptide Sequence | SEQ ID NO. |

Human

| | |
|---|---|
| MATNGSKVADGQISTEVSEAPVANDKPKTLVVKVQKKAADLPDRDTWKGRFDFLMSCVGYAIGLGNV | 2 |
| WRFPYLCGKNGGGAFLIPYFLTLIFAGVPLFLLECSLGQYTSIGGLGVWKLAPMFKGVGLAAAVLSF | |
| WLNIYYIVIISWAIYYLYNSFTTTLPWKQCDNPWNTDRCFSNYSMVNTTNMTSAVVEFWERNMHGMT | |
| DGLDKPGQIRWPLAITLAIAWILVYFCIWKGVGWTGKVVYFSATYPYIMLIILFFRGVTLPGAKEGI | |
| LFYITPNFRKLSDSEVWLDAATQIFFSYGLGLGSLIALGSYNSFHNNVYRDSIIVCCINSCTSMFAG | |
| FVIFSIVGEMAHVTKRSIADVAASGPGLAFLAYPEAVTGLPISPLWAILFFSMLLMLGIDSQFCTVE | |
| GFITALVDEYPRLLRNRRELFIAAVCIISYLIGLSNITGGGIYVFKLFDYYSASGMSLLFLVFFECV | |
| SISWFYGVNRFYDNIQEMVGSRPCIWWKLCWSFFTPIIVAGVFIFSAVQMTPLTMGNYVFPKWGQGV | |
| GWLMALSSMVLIPGYMAYMFLTLKGSLKQRIQVMVQPSEDIVRPENGPEQPQAGSSTSKEAYI | |

Mouse

| | |
|---|---|
| | 4 |
| MATDNSKVADGQISTEVSEAPVASDKPKTLVVKVQKKAGDLPDRDTWKGRFDFLMSCVGYAIGLGNV | |
| WRFPYLCGKNGGGAFLIPYFLTLIFAGVPLFLLECSLGQYTSIGGLGVWKLAPMFKGVGLAAAVLSF | |
| WLNIYYIVIISWAIYYLYNSFTTTLPWKQCDNPWNTDRCFSNYSLVNTTNMTSAVVEFWERNMHQMT | |
| DGLDKPGQIRWPLAITLAIAWVLVYFCIWKGVGWTGKVVYFSATYPYIMLIILFFRGVTLPGAKEGI | |
| LFYITPNFRKLSDSEVWLDAATQIFFSYGLGLGSLIALGSYNSFHNNVYRDSIIVCCINSCTSMFAG | |
| FVIFSIVGFMAHVTKRSIADVAASGPGLAFLAYPEAVTGLPISPLWAILFFSMLLMLGIDSQFCTVE | |
| GFITALVDEYPRLLRNRRELFIAAVCIVSYLIGLSNITGGGIYVFKLFDYYSASGMSLLFLVFFECV | |
| SISWFYGVNRFYDNIQEMVGSRPCIWWKLCWSFFTPIIVAGVFLFSAVGMTPLTMGSYVFPKWGQGV | |
| GWLMALSSMVLIPGYMAYMFLTLKGSLKGRLGVMIQPSEDIVRPENGPEQPQAGSSASKEAYI | |

### SLC6A1 Mutation Knockin Mouse Models

Both mutations SLC6A1(A288V) and SLC6A1(S295L), for which the knockin mouse models have been created, have been extensively characterized in a previous study at the molecular level. The SLC6A1(A288V) mouse line was generated in collaboration with the University of Connecticut Health Center, and the SLC6A1(S295L) mouse line was generated by Shanghai Model Organisms (Shanghai Model Organisms Center, Inc. Cat. No. NM-KI-190014). Both mouse lines are developed with the CRISPR-CAS9 global knockin approach. Both mouse lines are maintained in the C57BL/6J mice (Jax Stock #000664). The mice used for experiments in the study were generated by breeding the heterozygous with the wildtype. Both male and female heterozygous mice were used for breeding for experiments in both mouse lines.

### Cloning of GABA Transporter 1

The plasmid cDNA encoding enhanced yellow fluorescent protein (EYFP)-tagged rat GAT-1 has been previously described. The coding region of rGAT-1 was inserted into pEYFP-C1 (Clontech, Palo Alto, CA). QuikChange Site-directed Mutagenesis kit was utilized to introduce the GAT-1 variants into a wildtype GAT-1 plasmid. The product was then amplified via polymerase chain reaction, transformed using DH5a competent cells, and plated. A clone was chosen and grown overnight. All the GAT-1 variants were confirmed by DNA sequencing. Both the wildtype and the variant cDNAs were prepared with Qiagen Maxiprep kit.

### Mouse Cortical Neuron and Astrocyte Cultures

Mouse cortical neurons were prepared from postnatal day 0 pups. The neurons used for experiments were at day 15-17 after culture. Mouse cortical astrocyte cultures were prepared from the postnatal day 0-3-day old pups as previously described. Briefly, the cortices of postnatal day 0-3 pups were dissected. The tissues were minced after removing the meninges and then digested with 0.25% trypsin for 10 minutes at 37° C. The cells were cultured in poly-L-lysine coated 100 mm² dishes and maintained in Dulbecco's Modified Eagle's Medium supplemented with 10% FBS and 1% penicillin/streptomycin. The astrocytes used for experiments were at passage 2.

### Human Patient Derived Neurons and Astrocytes and Transfections in Human Astrocytes

The corrected and patient cell lines were maintained in mTeSR, and the plates were coated with Geltrex (1 (Geltrex stock solution): 50 DMEM/F12)) overnight. Geltrex stock was prepared by adding 5 mL DMEM to the original tube (5 mL DMEM/F12+5 mL Geltrex) and aliquoted to 300 μL per tube and stored at −20° C. The media was refreshed daily for iPSCs. The differentiation of neural progenitor cells (NPCs) was induced by STEMdiff SMADi Neural induction kit from STEMCELL.

The cortical inhibitory neurons were prepared following the protocol in previous reports. The neurons were used for experiment at day 60-65 after differentiation. Neurons were validated by immunostaining with NeuN, DLX, synapsin, and synaptophysin. Neuronal differentiations were initiated from the NPCs at day 10 in dish of passage 2 of neural progenitor cells (NPCs) after neuronal induction. The 1^{st} passage from iPSCs for neural induction was taken as P₀. The differentiation of astrocytes was started from NPC day 5 at P1. The experiments were carried out after 27 days for astrocytes or 2 months for neurons after differentiation. For experiment of each condition, at least double or triple the dishes were used each time. The differentiation of astrocytes was initiated by using the Astrocyte medium (ScienCell) for about 30 days and were passaged at ˜70% confluence. The cells used for experiment were about ˜80 to ˜90% confluence.

iPSCs

iPSCs were cultured in 35 mm² dishes with mTeSR from Gibco. The dishes were coated Geltrex (1:50 DMEM/F12) overnight. iPSCs were passaged at ˜80% confluency with 0.5 mM EDTA in 1×PBS. iPSCs at passages 14-22 were used in the study.

### NPCs

NPCs were differentiated from iPSCs at passage 22-24 with differentiation of neural progenitor cells (NPCs) was induced by STEMdiff SMADi Neural induction kit from STEMCELL. iPSCs were seeded in a Geltrex coated dish at a density of ˜2×10⁶ cells/per one 35 mm² dish or 6 well plate. The medium was refreshed daily, and the cells were passaged at day 5-7. NPCs at Passage 1 which was at least 10-12 days after neural induction were used for this study.

### Astrocytes

Astrocytes were differentiated starting from day 5 of NPC passage 1 with the astrocyte culture medium from Sciencell (catalog 1801) following the protocol as previously described. The cell culture medium was refreshed every other day and the cells were passaged at ˜70% confluency. About 1.5×10⁵ cells were seeded in each 35 mm dish or each well in 6-well plate when passaging. The differentiation was continuous until the day 30 and all the astrocytes used in experiments in this study were at day 30 after differentiation.

### GABAergic Inhibitory Neurons

Cortical inhibitory GABAergic neuronal differentiation was based on previously described. The differentiation started from iPSCs at passage 24-28 and was comprised of three 3 major steps including neural induction, cortical specification, and GABAergic neuronal differentiation.

### Neural Induction

The same protocol used for generating NPCs for astrocytes was used for neuronal differentiation.

### Cortical Specification

At day 10-12 of neural induction, the cells were harvested with accutase (Sigma), collected by centrifugation, washed in DPBS, and subsequently seeded at a density of 3×10⁵ cells in a 35 mm² dish or 6-well plate coated with poly-L-ornithine (PLO)/laminin (LMN; 20 μg/mL). In the condition involving cover slips, 3 cover slips were placed in each 35 mm² dish. From day 10-12, NPCs were patterned towards cortical fate using NMM medium (242 mL neurobasal, 242 mL DMEM/F12+Glutamax, 2.5 mL non-essential amino acids, 3.75 mL 100× Glutamax, 5.0 mL penicillin-streptomycin, 2.5 mL of 100×N2 supplement, 10 mL 50×B27, 312.5 μL insulin supplemented with 5 nM recombinant mouse sonic hedgehog (SHH C25II; RnD Systems), 1 μM SHH agonist purmorphamine (Miltenyi biotech), 10 ng/ml recombinant human brain derived neurotrophic factor (BDNF; Thermo-Fisher Scientific), 200 μM ascorbic acid (Sigma) and 100 μM 2′-O-di-butyryladenosine 3,5′-cyclic monophosphate (cAMP; Sigma). Media was refreshed on every 3^{rd} day.

### Maintenance and Further Differentiation

After 10 days, cells started to obtain NPC morphology and were gently harvested for further maturation. Briefly, cells were plated onto PLO/LMN coated coverslips/dishes in NMM medium supplemented with BDNF, ascorbic acid and cAMP. Medium was changed twice a week from this point onwards until day 60-65 starting from differentiation which is the age of the cells we use for this study. NMM media consist of 242 mL Neurobasal; 242 mL DMEM/F12+Glutamax; 2.5 mL Non-Essential Amino Acids; 3.75 mL 100× Glutamax; 5.0 mL penicillin-streptomycin; 2.5 mL 100×N2 Supplement; 5.0 mL 50×B27 Supplement; 312.5 μL Insulin (4 mg/mL stock to final concentration of 2.5 μg/mL); 1.75 μL BME (14.3 M stock to final concentration of 50 μM).

Neurons were coated with PLO (poly-L-ornithine, Sigma, 0.5 mg/mL in Borate buffer) for overnight and then Laminin (20 μg/mL) coating for at least 1 hr. Cell density at seeding: for iPSCs: 2-2.5×10⁶ cells per 35 mm² dish or 6 well; for NPCs: 2-2.5×10⁶ cells per 35 mm² or 6 wells; for astrocytes: 1.5×10⁵ cells per 35 mm² dish or 6 well. For neurons: ˜1 million cells/per 35 mm² dish or 6 wells.

### Culturing Human Embryonic Kidney (HEK293T) Cells and Chinese Hamster Ovary (CHO) Cells HEK 293T Cells

Human embryonic kidney cells were cultured as monolayers in 100 mm² dishes. Cells were cultured in Dulbecco's Modified Eagle's Medium supplemented with 10% FBS and 1% penicillin/streptomycin in 37° C. in 5% CO₂/95% air. Cells were maintained in culture medium until appropriate confluency for experimentation.

### CHO Cells

Chinese hamster ovarian cells were cultured as monolayers in 100 mm² dishes. Cells were cultured in Dulbecco's Modified Eagle's-F12 Medium supplemented with 10% FBS and 1% penicillin/streptomycin in 37° C. in 5% CO₂/95% air. Cells were maintained in culture medium until appropriate confluency for experimentation.

### Culture and Transfection for Mouse Neurons and Astrocytes

Mouse cortical neurons: Mouse neurons were cultured from postnatal day 0 mouse pups. The detailed procedures for neuronal culture have been previously described. The neurons were plated at a density of ˜0.5×10⁵ per well in the six-well plate or 35 mm² dish in plating media that contained 420 mL DMEM, 40 mL F12, 40 mL fetal bovine serum, 1 mL penicillin and streptomycin, and 0.2 mL L-glutamine (200 mM) for 4 hrs. Neurons were then maintained in Neurobasal media that contained B27 supplement (50:1), L-glutamine (200 mM), and 1 mL penicillin and streptomycin. The experiments for GABA uptake were directly conducted in the cultured six-well plate or the 35 mm² petri dishes.

### Mouse Cortical Astrocytes

Briefly, the dishes are coated with poly-lysine (0.1 mg/mL) for 2 hrs at room temperature. The cortices of postnatal day 0-3 pups sometimes from the same litter used for neuronal cultures were dissected. The tissues were minced after removal of meninges and then digested with 0.25% trypsin for 10 min at 37° C. The tissues were then mechanically dispersed with a 10 mL sterile pipette and a 1000 μL tip. The mixed cell suspension was then precipitated for 3 min in a 15 mL conic tube to remove the large chunks and cell debris. The mixed cell suspension was carefully aspirated and removed to another 15 mL conic tube with avoidance of the large chunks and debris. The cell suspension was centrifuged at 15000 rpm and the pellet was resuspended and seed at a density of ˜2-2.5 million/per 100 mm² dish. The media was changed in 24 hours. The cells were then maintained in Dulbecco's Modified Eagle's Medium (DMEM) supplemented with 10% FBS and 1% penicillin/streptomycin. The medium was refreshed every 4-5 days and passaged at 90-100% confluency. The astrocyte cultures at passage 0-2 were cultured in 100 mm² dishes and split into 60 mm² dishes or 35 mm² dishes for experiments. Transfection of the GAT-1 mutants in astrocytes were carried out with PEI in astrocytes at passage 2. The total CDNAs is 9 μg for a 100 mm² dish, 3 μg for a 60 mm² dish and 1 μg for a 35 mm² dish at a ratio of 1 μg:2.5 μL PEI. The cells were harvest for experiments after 48 hrs.

### Radioactive ³H-labeled GABA Uptake Assay

Briefly, cells were cultured in 5 mm² dishes for 3 days before the GABA uptake experiment in DMEM with 10% fetal bovine serum and 1% penicillin/streptomycin. The cells were then transfected with equal amounts of the wildtype or the mutant GAT-1 cDNAs (1 μg) for each condition at 24 hrs or 48 hrs after plating. GABA uptake assay was carried out 48 hrs after transfection. The cells were incubated with preincubation solution for 15 min and then incubated with preincubation solution containing 1 μci/mL 3H GABA and 10 μM unlabeled GABA for 30 min at room temperature. After washing, the cells were lysed with 0.25 N NaOH for 1 hr. Glacial acetic acid was added, and lysates were then determined on a liquid scintillator with QuantaSmart. The flux of GABA (pmol/μg/min) was averaged with at least triplets for each condition at each transfection. The average counting was taken as n=1. The untransfected condition was taken as a baseline that was subtracted from both the wildtype and the mutant conditions. The pmol/μg/min in the mutant was then normalized to the wildtype from each experiment, which was arbitrarily taken as 100%. The protocols for GABA reuptake assay in cultured mouse astrocytes, neuron or iPSC derived cells were modified from the GABA uptake protocol on HEK 293T cells. GAT-1 inhibitors CL-966 and NNC-711 were applied each time to make sure the radioactive counts were specific.

### Measurement of Surface and Total Expression of GAT-1 Using Flow Cytometry

Briefly, HEK293T cells were transfected using PEI reagent (40 kDa, Polysciences) at a DNA: transfection reagent ration of 1:2.5 and harvested 48 hours after transfection. To express wild-type (GAT-1) and mutant GAT-1, a total of 3 μg of subunit cDNAs was transfected into 60 mm² dishes. The transfected HEK293T cells were removed from the dishes by trypsinization and then re-suspended in FACS buffer (phosphate buffered saline (PBS) supplemented with 2% FBS and 0.05% sodium azide). Following washes with FACS buffer and permeabilization with Cytofix/cytoperm (BD Biosciences, CA) for 15 minutes, cells were incubated with rabbit polyclonal anti-GAT-1 antibody (1:200) (Synaptic System, catalog no. 274 102) for 2 h. Cells were then washed with FACS buffer and then incubated with fluorophore Alexa-555 or Alexa 488 conjugated goat anti-rabbit secondary antibody (1:400) for 1 h at 4° C. Cells were then washed with FACS buffer, and the cell surface fluorophore intensity was determined using a 3-laser LSR II machine at Vanderbilt Flow Cytometry Core. The acquired data was analyzed using FlowJo 7.1 (Tree Star, Inc., OR). Both the surface and the total GAT-1 protein were probed with rabbit polyclonal anti-Rabbit GAT-1, and 10,000 single cells were evaluated. The empty vector pcDNA mock transfected cells and untransfected cells were included as reference each time.

### PBA Administration In Vitro and In Vivo

For PBA administration in cells, the stocking solution of PBA (2 Mol) was dissolved in DMSO. A series of concentrations and incubation periods for PBA have been tested and the most optimal dosage (2 mM) and duration (24 hr) were identified and used throughout the study. For PBA administration in mice, the dose was chosen based on a dose-response experiment performed using increasing doses of PBA from 100 to 800 mg/kg to determine the most efficacious dose. Mice of both sexes at 2-4 months of age were dosed with PBA (100 mg/kg, i.p.) or vehicle for 7 days. PBA solution was prepared by dissolving PBA in 0.9% normal saline and then titrating equimolecular amounts of 4-phenylbutyric acid (Sigma, Madrid, Spain) and 1 Mol potassium hydroxide to pH 7.4. The working solution was stored at 4° C.

### Synchronized Video-Monitoring EEG Recordings

The surgery to implant the EEG head mount and the video-monitoring synchronized EEG recordings were conducted as described by Kristensen et al., Pharmcol. Rev. 63 (3): 585-640 (2011) and Cai et al., Exp. Neurol. 3:112973 (2019).

EEG Analysis with Seizure Pro Software

For recordings, the sampling rate was set at 400 Hz with a pre-amplifier gain of 100 Hz. EEG and EMG channels had a filter set at 25.00. EEG recordings were scored blindly by a skilled scorer using the Sirenia Seizure Pro software. A power analysis was performed using the theta frequency band of 5-7 Hz. The 5-7 Hz SWDs band was chosen because it is the mouse correlate of 2-4 Hz SWDs in humans. An average power was calculated using baseline recordings and applied to seizure analysis for treatment recordings. Seizures identified by the software were confirmed using video recordings of the period. The Racine scale was used for seizure identification (Stage 1: mouth/facial movements; Stage 2: head nodding; Stage 3: forelimb clonus; Stage 4: rearing; Stage 5: rearing and falling). The identified SWDs were then confirmed with video monitoring and compared across treated and non-treated recordings. The total seizure events over 48 hrs of EEG recordings were reported.

### Statistical Analysis

Data were expressed as mean±SEM. Proteins were quantified by Odyssey software and data were normalized to loading controls and then to the wildtype subunit protein, which was arbitrarily taken as 1 in each experiment. Fluorescence intensities from confocal microscopy experiments were determined using MetaMorph imaging software, and the measurements were carried out in ImageJ. For statistical significance, one-way or two-way analysis of variance (ANOVA) with post hoc Dunnett or Newman-Keuls test were used. In some cases, unpaired t-test or one sample t-test was performed (GraphPad Prism, La Jolla, CA), and statistical significance was taken as p<0.05.

### Example 2

### Missense and Nonsense SLC6A1 Variants Caused Partial or Complete Loss of GABA Uptake Function Due to Reduced GAT-1 Surface Protein Expression

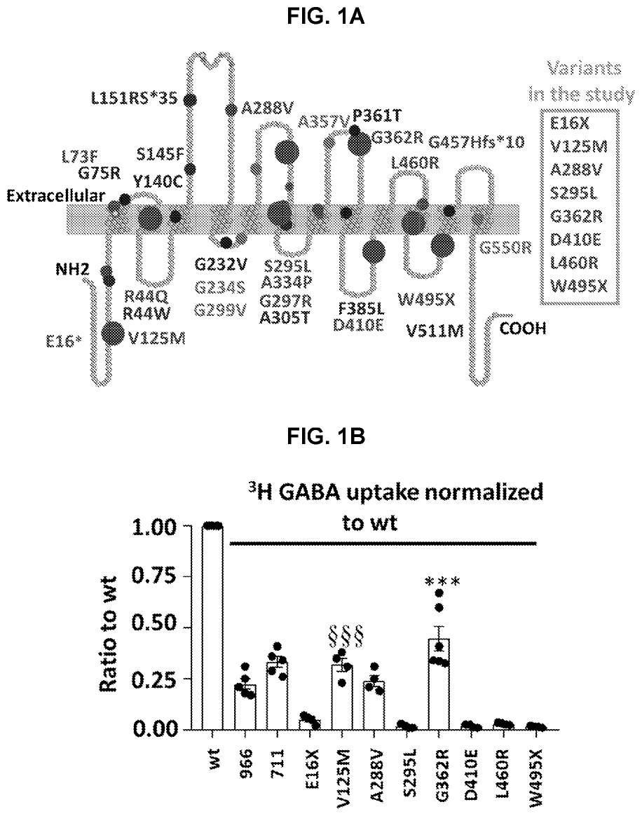

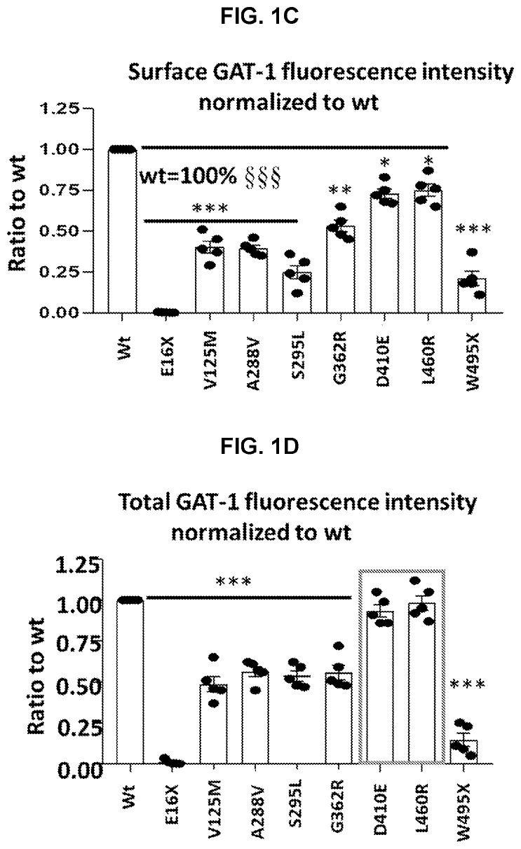

SLC6A1 variants are distributed in various locations of the GAT-1 protein peptide and are associated with various epilepsy syndromes and neurodevelopmental disorders. Eight mutations were selected for the study as they are representative of missense and nonsense mutations with premature stop codon mutations generated at the N or C-terminus (FIG. 1A). HEK293T cells were transfected with the empty vector pcDNA, wildtype, or the mutant GAT-1^{YFP} for 48 hrs before GABA uptake assay. All mutations showed reduced GABA uptake activity (FIG. 1B), levels similar to those observed in cells expressing the wildtype GAT-1 treated with GAT-1 inhibitors CI-966 (50 μM) or NNC-711 (35 μM) (FIG. 1B). Consistently, the cell surface expression of the GAT-1 was reduced in the mutant (FIG. 1C). However, the expression of GAT-1 was reduced in some mutations but not in the D410E and L460R mutations.

It is interesting that the mutations resulted in loss of GABA uptake function regardless of whether they were missense or nonsense variants in the N-terminus or C-terminus (FIG. 1D). In all surveyed variants, the GABA uptake was less than 50% of the wildtype. All eight variants had reduced surface expression with varying magnitude as evaluated by a high-throughput assay, flow cytometry (0.002 for E16X, 0.402 for V125M, 0.394 for A288V, 0.248 for S295L, 0.532 for G362R, 0.73 for D410E, 0.75 for L460R and 0.21 for W495X vs. wt=1). Most mutations had reduced total protein expression except D410E and L460R (0.01 for E16X, 0.488 for V125M, 0.562 for A288V, 0.536 for S295L, 0.558 for G362R, 0.93 for D410E, 0.98 for L460R and 0.146 for W495X). This suggests that there is a correlation in the reduction of surface protein and total protein but no direct correlation to the GABA uptake function, as some mutant protein is trafficking competent but is nonfunctional or has altered function.

### SLC6A1 Mutations Produced Less Mature GAT-1 Protein but More ER Retained Immature GAT-1 Protein

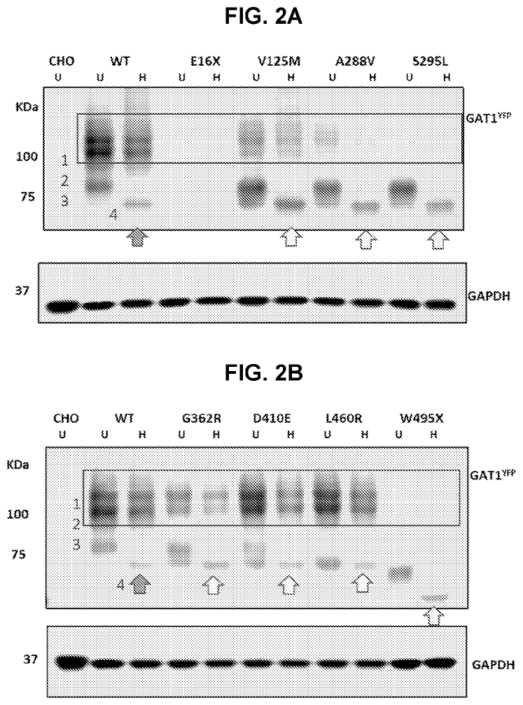

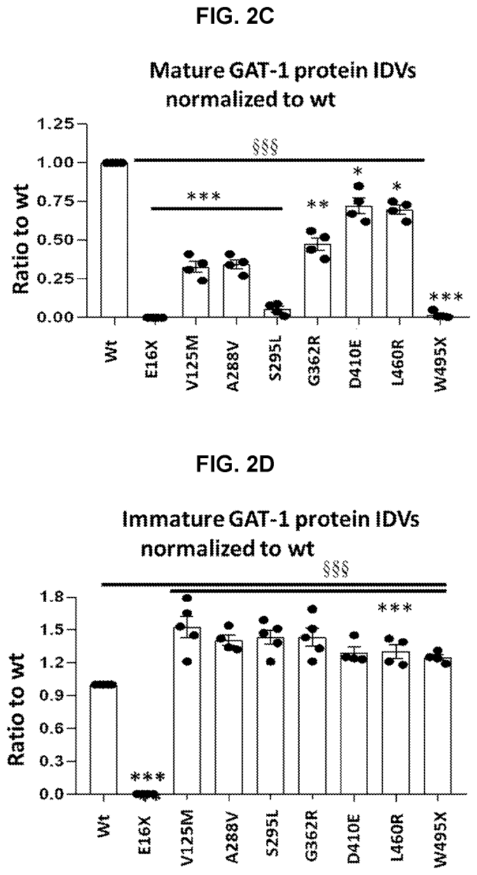

The reduced GABA uptake activity could be caused by reduced cell surface expression or the altered gating kinetics of the transporter channels. Based on studies with SLC6A1 and GABA_{A} receptor epilepsy mutations, the reduction in cell surface expression of the mutant protein is the major mechanism and is mainly due to ER retention of the misfolded mutant protein, while the altered gating is a minor mechanism. Based studies with GABA_{A} receptors, only those proteins that are mature for glycosylation and that have trafficked beyond the ER to reach the cell surface and synapse can function. The maturity of the mutant GAT-1 was determined with Endo-H digestion, which removes the ER added glycan but not the glycan added beyond ER. The GAT-1^{YFP} protein runs with 3 bands (band 1, 2 and 3) at 108 kDa, 96 kDa, and 90 kDa respectively (FIGS. 2A-B). Endo-H treatment removes the glycan added in ER but not those added beyond ER. Thus, the ER retained protein run at a lower molecular mass (band 4). The band 1 and band 2 are classified as mature forms of GAT-1 while the band 3 and the down-shifted band 4 are classified as immature GAT-1. The GAT-1(E16X) mutant protein was undetectable likely because of fast degradation of the small peptide due to the early premature stop codon. The GAT-1 (W495X) mutation ran at a more reduced molecular mass due to the premature stop codon generated by the nonsense mutation (FIG. 2B). Most of the mutant GAT-1 had reduced mature form of GAT-1 (0.00 for E16X, 0.327 for V125M, 0.345 for A288V, 0.05 for S295L, 0.475 for G362R, 0.72 for D410E, 0.69 for L460R and 0.019 for W495X) but increased Endo-H sensitive immature form of GAT-1 (0.00 for E16X, 1.526 for V125M, 1.405 for A288V, 1.432 for S295L, 1.434 for G362R, 1.293 for D410E, 1.30 for L460R and 1.248 for W495X). (FIGS. 2C-D), suggesting ER retention of the mutant transporter.

Levels of the mature form of GAT-1 in GAT-1(V125M, A288V, S295L and S295L and G362R) are correlated with the level of GABA uptake function. The mature form of GAT-1(D410E) and GAT-1(L460R) in HEK 293T cells does not correlate with the GABA uptake function. The GAT-1(E16X) and GAT-1(W495X) had almost no mature GAT-1 protein (FIG. 2C). By contrast, the immature GAT-1 shifted with Endo-H was higher in all surveyed mutant GAT-1 except GAT-1(E16X) as the short, truncated protein peptide in the mutant protein may be subjected to fast disposal inside ER. It is worth noting that the immature form of the mutant GAT-1 for V125M, A288V, S295L, G362R, D410E and L460R either displays a smear or run at a lower molecular mass, suggesting delayed glycosylation due to protein misfolding.

### PBA Increased GABA Uptake in the Wildtype and the Mutant Transporters in HEK293T Cells

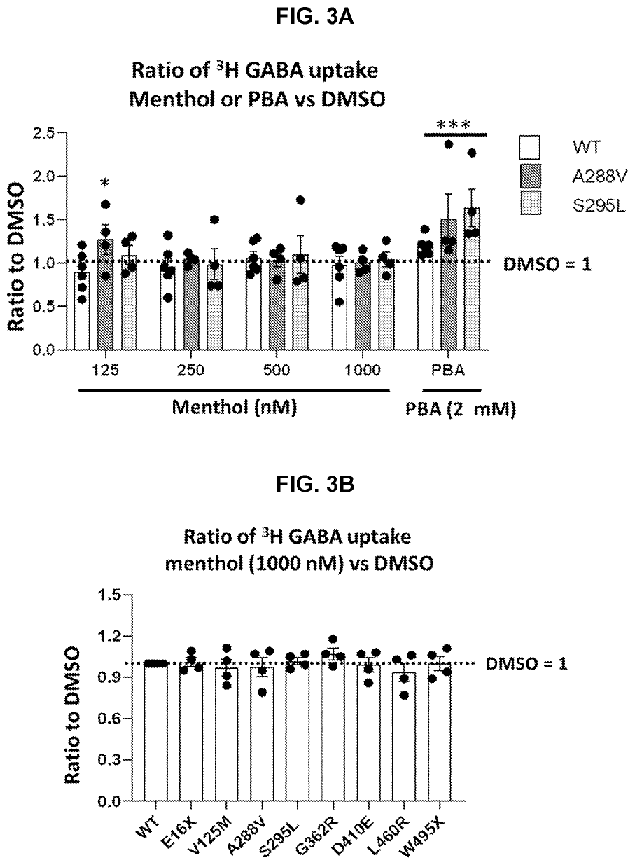

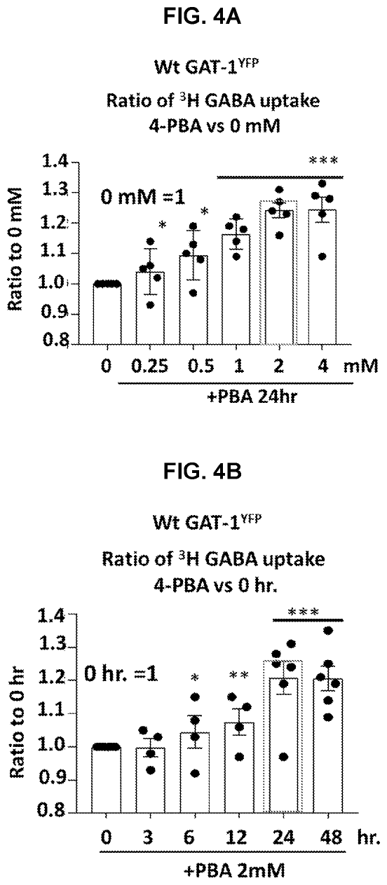

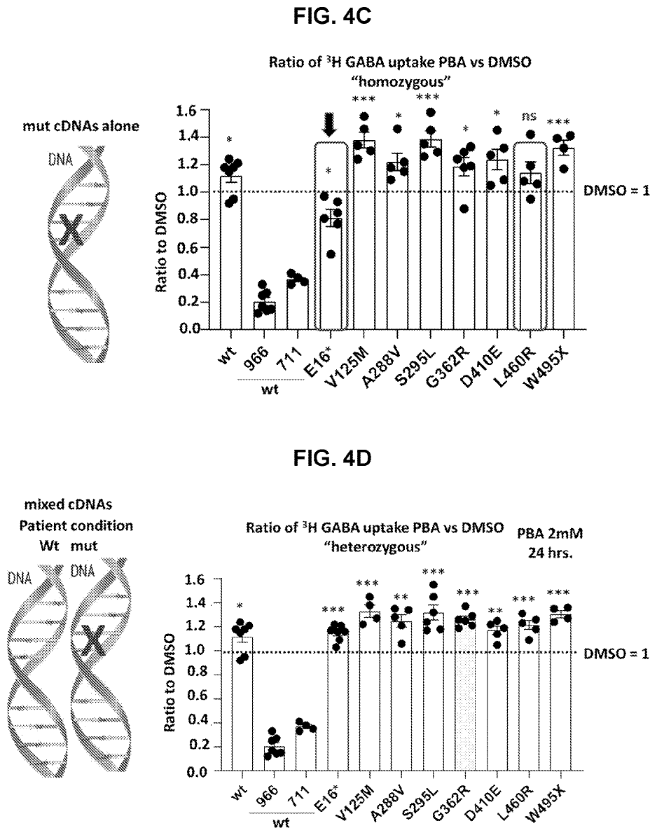

Previously studies demonstrated that ER retention of the mutant protein can exacerbate the disease phenotype by preventing the efficient trafficking of the wildtype subunits. PBA was compared with other small molecules or chaperones such as menthol that could potentially modulate protein trafficking and increase GABA uptake (FIGS. 3A-C). PBA was determined to be effective for restoring GABA uptake among other tested compounds. The effect of PBA on rescuing the wildtype and the mutant GAT-1 activity were analyzed. The effect of PBA on the wildtype GAT-1 was determined first. The HEK 293T cells were transfected with GAT-1^{YFP} for 48 hrs and PBA was applied with a series of concentrations from 0 to 4 mM (FIG. 4B) and time durations from 0 to 48 hrs (FIG. 4A) before evaluation. PBA concentration and time dependently increased the GABA uptake activity (FIG. 4A-B). However, the effect of PBA at 2 mM and 24 hrs was the most optimal (1.24±0.05 for 24 hrs and 1.24±0.09 for 4 mM vs. wildtype 0), as occasional cell loss was observed in cells treated with PBA at 4 mM and/or 48 hrs. A PBA treatment of 2 mM and 24 hrs was used for the following experiments.

Since all patients carrying SLC6A1 variants are heterozygous and only one allele is affected in patients as illustrated (FIG. 4C), the effect of PBA in the cells expressing the mutant cDNAs alone was tested (“homozygous”) (FIG. 4C) or a mixture of the wildtype and the mutant CDNAs (“heterozygous”) (FIG. 4D). The cells expressing the mutant alone, PBA did not increase GABA uptake for the GAT-1(E16X) and GAT-1(L460R) mutations (0.813±0.06 for E16X, 1.378±0.056 for V125M, 1.22±0.063 for A288V, 1.386±0.058 for S295L, 1.185±0.065 for G326R, 1.236±0.074 for D410E, 1.14±0.07 for L460R, and 1.323±0.054 for W495X) (FIG. 4C). However, PBA increased the mutant GAT-1 activity in all tested variants except the GAT-1(E16X) in the “heterozygous” condition, which reflects the patient condition in which a wildtype allele is present alongside the mutant allele (1.149±0.025 for E16X, 1.33±0.05 for V125M, 1.248±0.053 for A288V, 1.318±0.062 for S295L, 1.262±0.026 for G362R, 1.17±0.035 for D410E, 1.214±0.037 for L460R and 1.305±0.0296 for W495X) (FIG. 4D). The failure to increase GABA activity in GAT-1(E16X) when expressed alone is most likely because of the very short peptide resulting from the early truncation. This renders it very misfolded while lacking ability to be refolded, thus being subjected to quick degradation inside the ER. In the “heterozygous” condition, in which the cells are transfected with mixed wildtype and mutant GAT-1 cDNAs, all the mutant conditions had increased GABA uptake activity compared with the DMSO treated conditions. This is likely due to the increased wildtype allele function. This suggests that PBA can have a broad application across an array of variants when in the heterozygous state. It is thus possible that all the patients can benefit from PBA, even in a case like GAT-1(E16X) when the mutant protein cannot be rescued.

PBA Increased GABA Uptake in the Patient iPSC Derived Astrocytes and Neurons

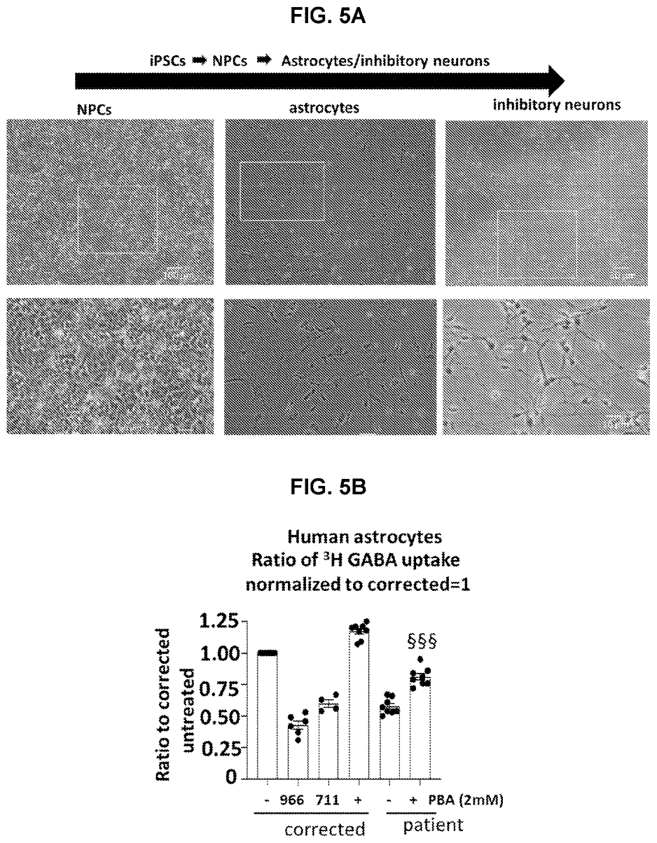

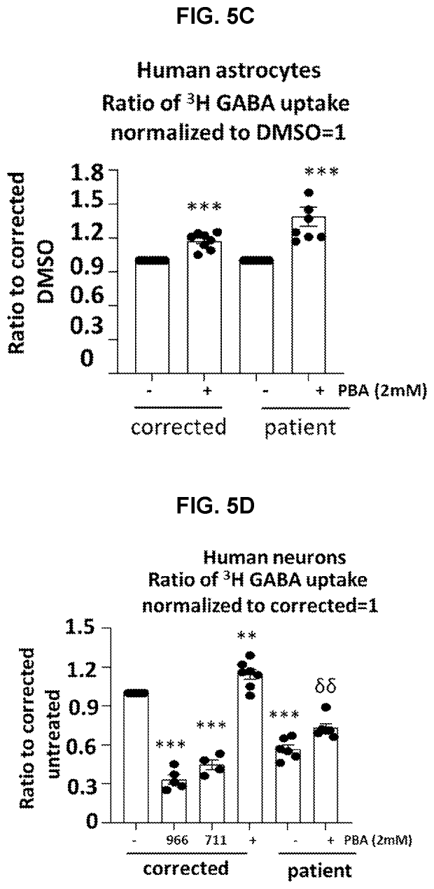

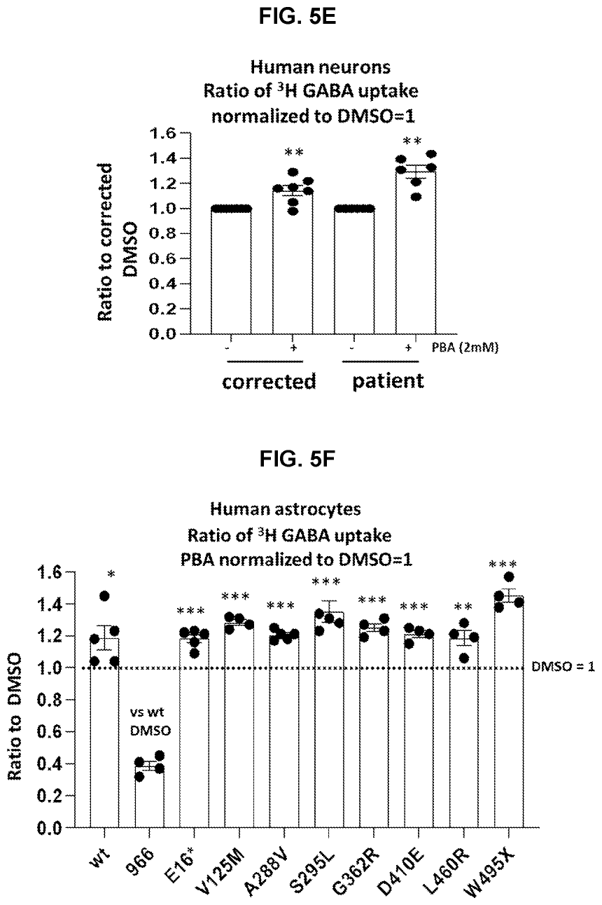

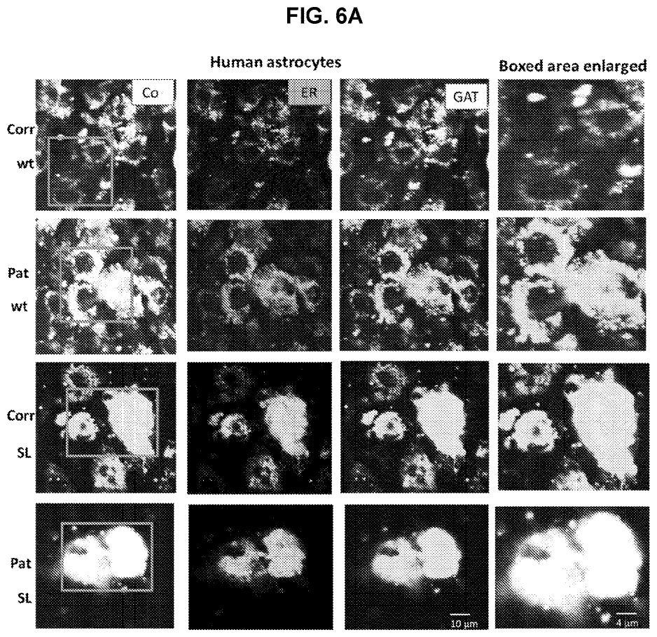

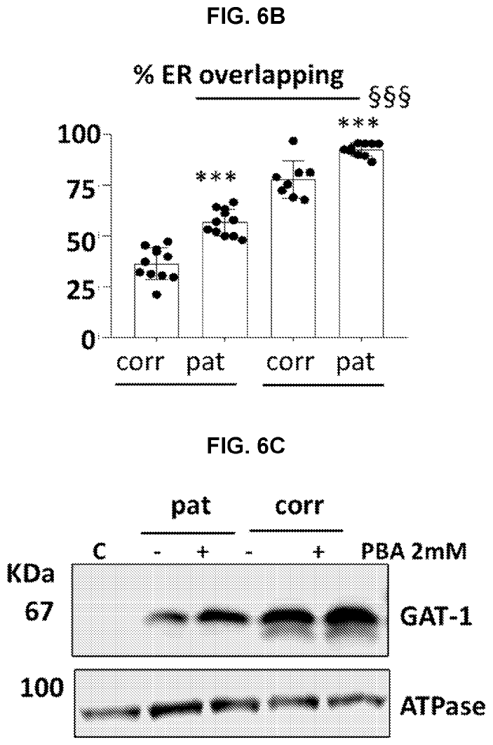

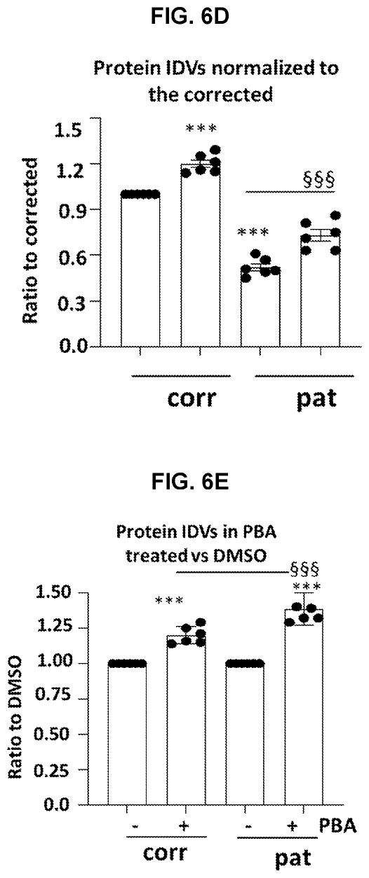

Experiments were used to determine whether PBA could restore the GAT-1 activity in human patient iPSC-derived astrocytes and neurons. Astrocytes were used as there is a direct correlation of astrocytic GAT-1 deficit with thalamic absence seizures. iPSCs were differentiated to neuronal progenitor cells (NPCs) and then differentiated the NPCs into astrocytes or inhibitory neurons. Human astrocytes derived from iPSCs around 30 days after differentiation were treated either with DMSO or PBA (2 mM) for 24 hrs before the GABA uptake assay. Based on the protocol, >90% of astrocytes adopted typical star-like astrocytic morphology after day 27 of differentiation (FIG. 5A). Similarly, after 2 months of differentiation, ˜90-95% of cells adopted typical GABAergic interneuron morphology. When compared with the corrected isogenic control cell line, the patient astrocytes showed reduced GABA uptake activity. However, PBA (2 mM) treatment for 24 hrs increased the activity in both isogenic cells and patient cells, increasing the uptake of the patient cells from 57% to 81% of the corrected cells (FIG. 5B). The increased magnitude is larger in the mutant than the isogenic control astrocytes (1.173±0.026 for control vs. 1.387±0.08 for patient) (FIG. 6C).

Then the effect of PBA in human iPSC-derived neurons was determined (FIGS. 5D-E) and identified a ˜20% increase of GABA uptake in PBA treated neurons. Since PBA caused similar upregulation on GABA uptake in both astrocytes and neurons, astrocytes were used because of their direct involvement in seizure activity and tested the effect of PBA on all eight mutations in this study in human astrocytes.

Wildtype or the mutant GAT-1^{YFP} were transfected (1 μg cDNAs per 35 mm² dishes) in the astrocytes derived from the corrected iPSCs for 48 hrs. GABA uptake activity was determined in astrocytes treated with DMSO or 2 mM PBA for 24 hrs. GAT-1 inhibitor CI-966 (50 μM) or NNC-711 (35 μM) was applied to confirm that decreased radioactive uptake correlated with decreased GAT-1 functioning. PBA increased GABA activity in the wildtype and all mutant conditions (1.188±0.075 for wt; 1.182±0.025, E16X, 1.285±0.018 for V125M, 1.204±0.031 for A288V, 1.353±0.065 for S295L, 1.25±0.023 for G362R, 1.21±0.022 for D410E, 1.18±0.045 for L460R and 1.45±0.04 for W495X). However, the GABA activity was increased the most in the astrocytes expressing the mutant GAT-1(W495X) (FIG. 5F).

The Human iPSC Derived Astrocytes Expressing Mutant GAT-1(S295L) Caused Retention of the Wildtype GAT-1 Inside the ER, Suggesting a Dominant Negative Effect

Previously experiments demonstrated that GAT-1(S295L) was retained inside the ER. However, it is unknown if the mutant GAT-1 would suppress the wildtype GAT-1 via aberrant oligomerization of the wildtype GAT-1 with the mutant GAT-1. The GAT-1 expression pattern was compared in the corrected or the patient iPSC derived astrocytes. Human astrocytes derived from iPSCs 27-30 days after differentiation were cotransfected with the wildtype or the mutant GAT-1 cDNAs with the ER marker ER^{CFP} (FIG. 6A). To evaluate the subcellular localization of the mutant GAT-1, the colocalization fluorescence was measured for the GAT-1-representing YFP and ER-representing CFP in the corrected or the patient astrocytes coexpressing the wildtype GAT-1^{YFP} or the mutant GAT-1^{YFP} with ER^{CFP}. The mutant GAT-1(S295L) had an increased overlapping fluorescence signal with ER than the wildtype. Importantly, the wildtype GAT-1 expressed in the patient astrocytes had a greater ER overlapping fluorescence signal than the corrected cells (36.29%±2.4% for GAT-1^{YFP} in corrected cells, 56.6%±1.95% for GAT-1^{YFP} in patient cells, 77.78%±3.26% for GAT-1(S295L) YFP in corrected cells, 92.49%±0.99% for GAT-1(S295L) YFP in patient cells) (FIG. 6B). It is worth noting that there were fewer cells with positive yellow fluorescence signal despite the large lumps of YFP in the patient cells.

Then it was determined whether PBA could increase the GAT-1 protein expression in the human astrocytes and found that PBA treatment increased the GAT-1 expression in both the corrected and the patient conditions (1.20±0.024 for correct vs. 1.39±0.046 for patient) (FIGS. 6C-E). The total GAT-1 protein of the patient astrocytes was increased from 52.2% to 73.2% of the corrected. The surface GAT-1 expression could not be determined because of the low yield of protein in astrocytes. It is likely that the increased GAT-1 in the patient astrocytes is due in part to the wildtype GAT-1. More detailed characterization with specific tags in GAT-1 to distinguish the wildtype vs. the mutant allele may further elucidate the contribution to increased GAT-1 from each allele.

PBA Increased GABA Uptake in the Astrocytes and Neurons in SLC6A1^{+/A288V} and SLC6A1^{+/S295L} Knockin Mice

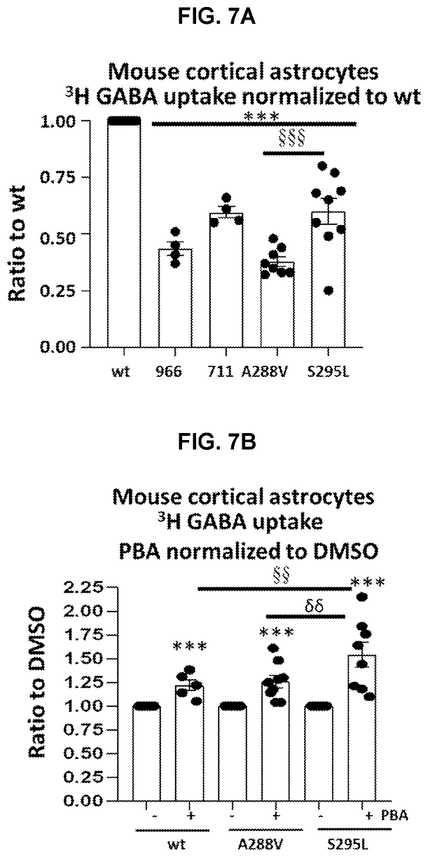

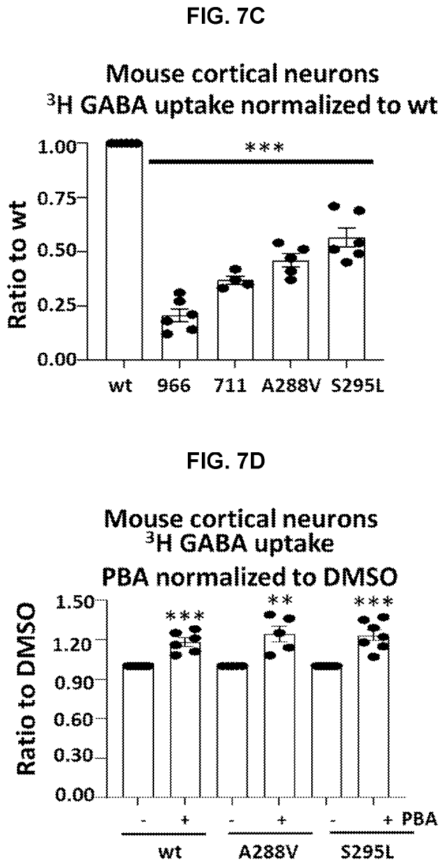

The effect of PBA in both astrocytes and neurons from both SLC6A1^{+/A288V} and SLC6A1^{+/S295L} knockin mice was evaluated. The mutants A288V and S295L were evaluated in mice due to the extensive characterizations of the two variants in vitro. Cortical neurons and astrocytes were used because the cortico-thalamic pathway is heavily involved in absence seizures. Additionally, the data from the cortical astrocytes and thalamic astrocytes was comparable (data not shown). The cortical tissue was used since the cortical tissue is more abundant than the thalamic tissue. GABA uptake activity can be impacted by both GAT-1 and GAT-3, so the cells were treated with a GAT-3 inhibitor, SNAP5114 (30 μM), to ensure only GAT-1 activity was measured. Compared with the wildtype, the astrocytes cultured from the heterozygous pups had reduced GABA uptake activity (0.38±0.021 for A288V and 0.60±0.06 for S295L) (FIG. 7A). PBA treatment increased the GABA uptake activity in the astrocytes cultured from the wildtype and the mutant pups (1.22±0.058 for wt; 1.26±0.063 for A288V and 1.54±0.13 for S295L) (FIG. 7B). A similar pattern of GABA uptake reduction in the mutant mice and the upregulation in PBA treated cells was observed in the cultured cortical neurons from the knockin mice (FIGS. 7C-D).

GAT-1 Protein Expression was Globally Reduced in SLC6A1^{+/A288V} and SLC6A1^{+/S295L} Knockin Mice and was Partially Restored by PBA

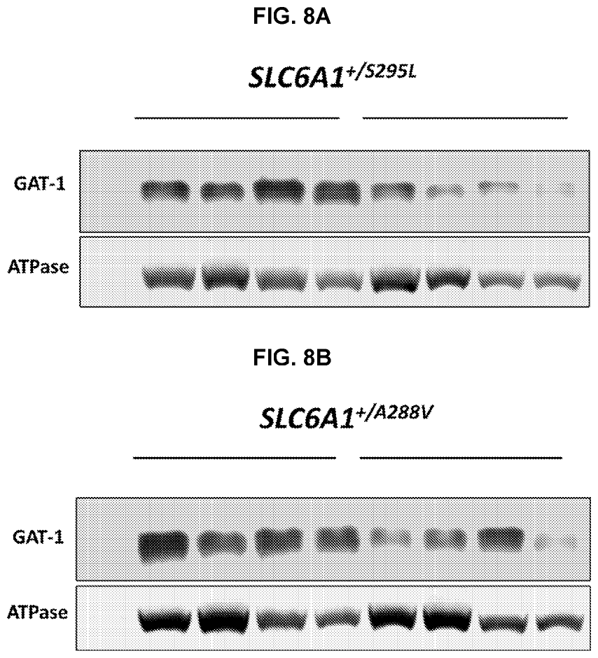

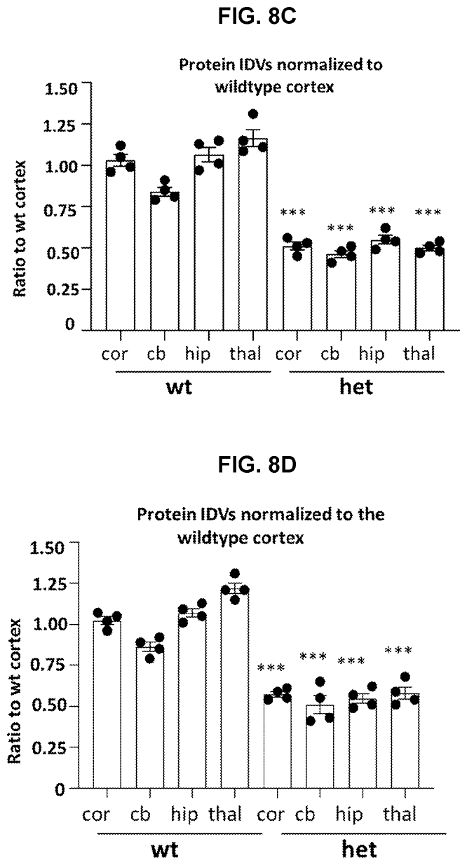

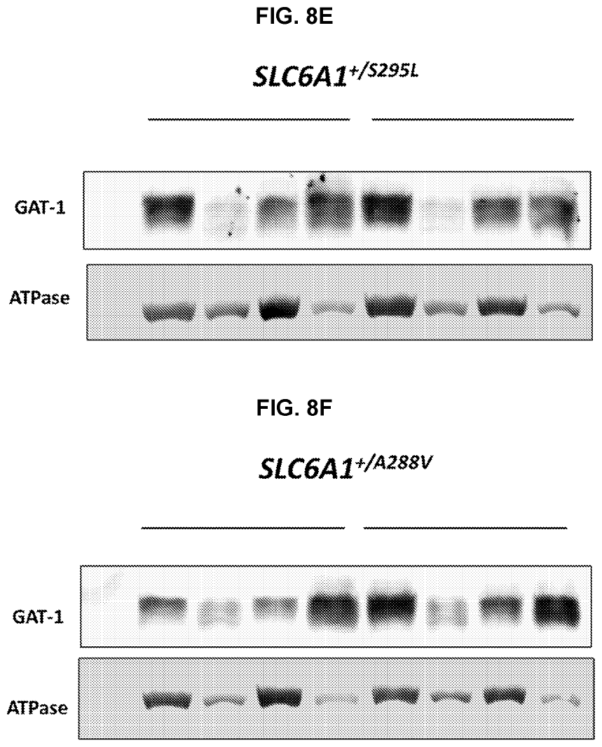

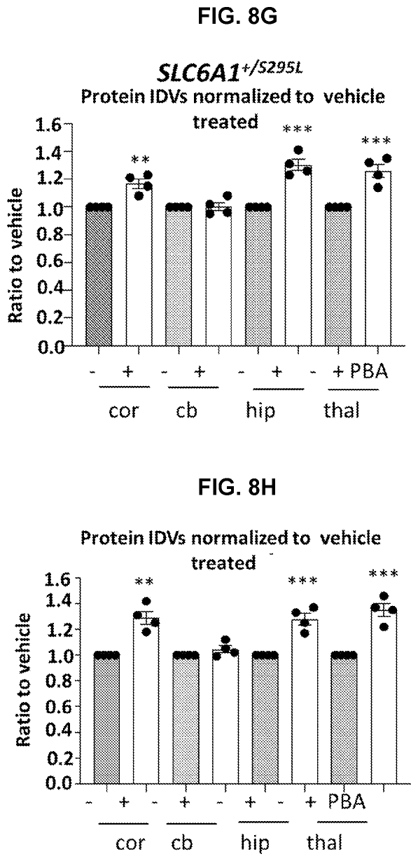

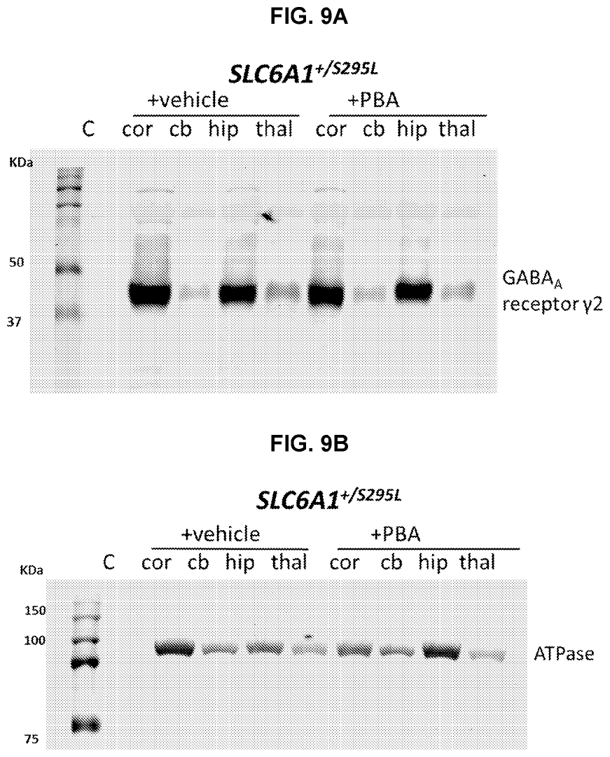

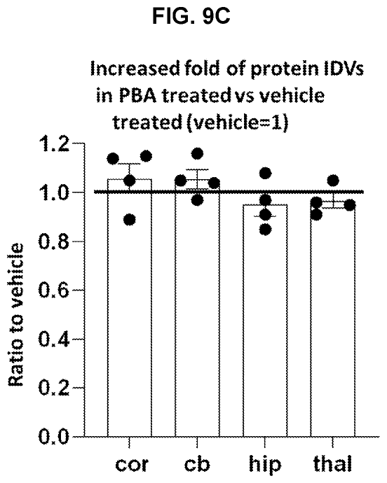

The increased GABA uptake activity could be due to increased GAT-1 protein expression caused by promoted membrane trafficking by PBA. It was then determined whether PBA alters the GAT-1 in knockin mice. The GAT-1 protein expression of the mutant mice was profiled. Then mice around 2 months old were treated with either saline or PBA for 1 week. The lysates from cortex, cerebellum, hippocampus, and thalamus were surveyed. Compared with the wildtype, the heterozygous mice from both the SLC6A1^{+/A288V} and SLC6A1^{+/S295L} mouse lines showed reduced GAT-1 expression in all surveyed brain regions (wt: 1.025±0.023 for cortex, 0.86±0.03 for cerebellum; 1.07±0.03 for hippocampus; 1.22±0.033 for thalamus; Slc61^{+/A288V} het: 0.57±0.0165 for cortex; 0.51±0.056 for cerebellum; 0.547±0.029 for hippocampus; 0.58±0.037 for thalamus; Slc61^{+/S295L} het: 0.513±0.023 for cortex; 0.465±0.021 for cerebellum; 0.55±0.023 for hippocampus; 0.50±0.016 for thalamus) (FIGS. 8A-B), consistent with previous findings in vitro that the GAT-1(A288V) and GAT-1(S295L) mutations cause ER retention of the mutant protein, consequently leading to enhanced degradation. Compared with vehicle treatment, PBA treatment increased GAT-1 expression in the cortex, hippocampus, and thalamus after normalization with the housekeeping protein ATPase. PBA increased the GAT-1 expression in the cortex, hippocampus, and thalamus, although this was not observed in the cerebellum (Slc61^{+/A288V} PBA: 1.29±0.05 for cortex, 1.045±0.028 for cerebellum; 1.28±0.044 for hippocampus; 1.35±0.049 for thalamus; S/c61^{+/S295L} PBA: 1.168±0.033 for cortex; 1.03±0.03 for cerebellum; 1.30±0.04 for hippocampus; 1.26±0.04 for thalamus vs. the same brain region of the vehicle treated mice taken as 1) (FIGS. 8C-D). This may suggest that the PBA-induced increase of GAT-1 can be region-specific and that the increased membrane trafficking of GAT-1 contributes to increased GABA uptake. It is worth noting that the PBA treatment did not increase GABA_{A} receptor γ2 subunit (FIGS. 9A-C), suggesting that the PBA-induced protein increase could be mutation-bearing gene specific, but this merits more elucidation.

### PBA Alone Reduced Seizure Activity in SLC6A1^{+/S295L} Knockin Mice

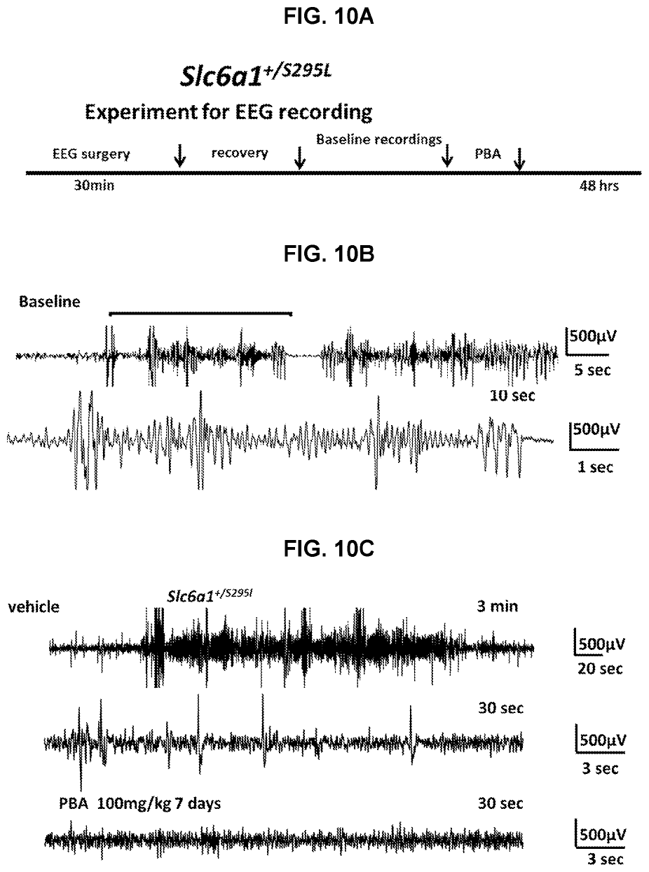

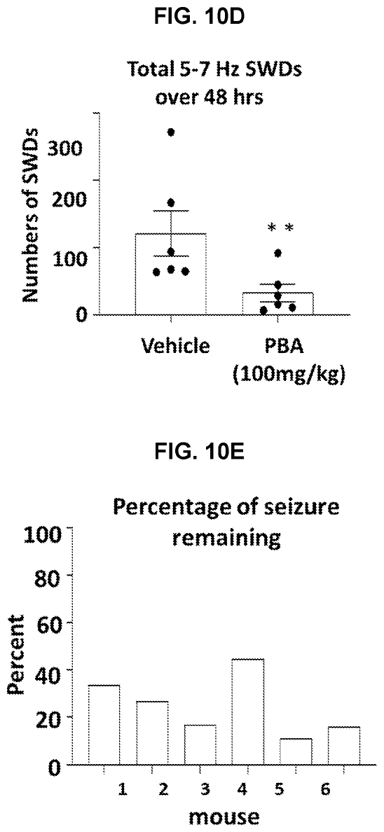

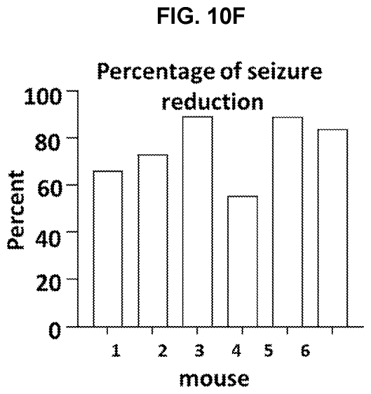

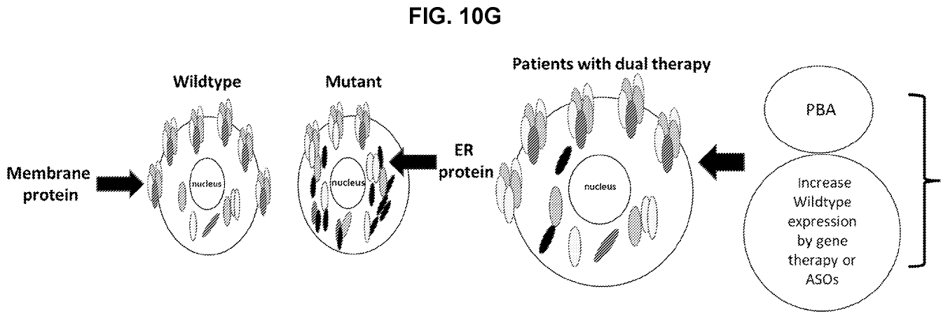

A major phenotype in SLC6A1 variant mediated disorders is epilepsy, leading us to investigate whether PBA reduces seizure activity in mice. The effect of PBA in SLC6A1^{+/S295L} mice were tested. First, ˜2-month-old mice were implanted with EEG head mounts. After 5-7 days recovery, the mice were treated with a normal saline vehicle for 7 days, and then subject to video monitored EEG recording for 48 hrs. After at least one day rest, the mice were administered with PBA (100 mg/kg) for 7 days followed by 48-hour EEG recording (FIG. 10A). Frequent absence-like seizures and occasionally generalized tonic clonic seizures were observed during routine handling in SLC6A1^{+/S295L} mice. In EEG recordings, the SLC6A1^{+/S295L} mice displayed frequent absence like activity (5-7 Hz) and some myoclonic jerks with or without behavioral correlation (FIG. 10B-C). However, compared with vehicle treated baseline recordings, PBA treatment reduced the 5-7 Hz SWDs from 127.7±34.08 with vehicle treated to 33.3±13.02 over 48 hrs EEG recordings (FIG. 10C, D). Importantly, every mouse had reduced seizures compared to vehicle treatment ranging from 55% to 89% seizure reduction and with 11% to 44.6% seizure remaining (FIG. 10E-F). This suggests PBA alone can reduce seizure activity in SLC6A1^{+/S295L} mice. This is likely due to promoted membrane protein forward trafficking and increased functional GAT-1 expression, which includes both the wildtype allele and the rescuable mutant allele. It is worth noting that there was no increase in the expression of GABA_{A} receptor subunits, such as the γ2 subunit. However, the level of specificity in the upregulation of the functional GAT-1 vs. other membrane proteins merits more detailed investigation.

### Reduced Membrane Trafficking Due to ER Retention is a Major Cause for SLC6A1 Variant Mediated Disorders

SLC6A1 variants are associated with a wide spectrum of neurodevelopmental disorders. This work has studied the impact of the variants on GABA uptake function, membrane trafficking, and subcellular localizations of the mutant protein in both cultured neurons and astrocytes from mouse and human iPSC derived GABAergic neurons and astrocytes. The mutant GAT-1 stemming from missense mutations resulted in complete or partial loss of GABA uptake function while the premature codon generating nonsense mutations resulted in nearly complete loss of function. The mutant protein is often retained inside the ER and subject to enhanced degradation, as observed in many GABA_{A} receptor subunit variants, suggesting that SLC6A1 variants and GABA_{A} receptor subunit variant mediated disorders could be rescued with a common treatment targeting ER retention.

### The Impaired Membrane Trafficking Resulted in Reduced Functional and Mature GAT-1 but Increased Nonfunctional, Immature GAT-1

The molecular mechanisms underlying the reduced surface expression of the mutant GAT-1: For a membrane protein such as GAT-1 or GABA_{A} receptor subunits, only those proteins that are correctly folded and fully glycosylated can traffic to the cell surface or synapse where it exerts biological function. The protein that is left inside the ER thus has no biological function. However, the ER retained mutant protein can prevent the wildtype protein from correct oligomerization and trafficking, causing ER stress in the cell. Endo-H digestion can distinguish the mature form of GAT-1 vs. immature GAT-1 because the ER-attached glycan is sensitive to Endo-H digestion. Based on the findings, the mutant GAT-1 resulted in decreased functional mature GAT-1 levels but increased amounts of immature ER-retained GAT-1 except for GAT-1(E16X), which is likely subject to rapid disposal. Similar ER retention of GAT-1 was reported in astrocytes in previous studies. Therapeutic strategies that increase mature GAT-1 levels and reduce the immature GAT-1 levels should be beneficial for many mutations with this underlying mechanism.

### The Mutant GAT-1 can Prevent Forward Trafficking of Wildtype GAT-1

The oligomerization status and detailed structure of membrane-bound GAT-1 remain unknown. It is possible that wildtype GAT-1 and mutant GAT-1 can form dimers or other high molecular mass protein complexes and that the trafficking-deficient mutant GAT-1 could potentially interfere with the trafficking of the wildtype. Thus, any drug that can promote protein trafficking could be beneficial to SLC6A1 variant-mediated disorders and can be considered for further treatment development. This notion is evidenced in the human astrocytes, where wildtype GAT-1 was more ER bound when expressed in the patient cells than expressed in the corrected cells (FIG. 6). The GAT-1(S295L) protein is retained inside the ER with minimal surface expression. In the patient cells expressing the mutant GAT-1(S295L) YFP, the GAT-1-representing yellow fluorescence formed large clumps inside cells with enlarged ER, suggesting substantial ER retention of the mutant protein inside the ER. PBA increased GABA uptake activity and the total functional GAT-1 protein in both the corrected and patient astrocytes.

### PBA Could Rescue the GABA Uptake Activity Across Cell Types

The effect of PBA was tested in heterologous cells, neurons, and astrocytes from both mice and humans finding that PBA increased GABA uptake across cell types. It is worth noting that an increase of GABA uptake in neural progenitor cells after PBA treatment was observed, although the baseline activity of GABA uptake in NPCs is relatively low (data not shown). This is consistent with a previous study on comparison of GABA uptake in NPCs, astrocytes, and inhibitory neurons. This suggests that the protein quality control machinery is conserved across species and cell types. This is important considering the early onset of the SLC6A1 variant-mediated disorders in human patients and the expression of GAT-1 function in multiple cell types. This indicates PBA treatment can increase GAT-1 functioning before and after the postmitotic mature neurons are formed. It also demonstrates that PBA can improve the function of GAT-1 in both progenitor cells and the derived neurons and astrocytes. These data indicate that PBA treatment is likely disease modifying, as it can improve GAT-1 function in all involved cell types. It corrects the patho-mechanisms at a root level instead of simply masking disease symptoms, thus enable it to mitigate both seizures and comorbidities while improving disease outcome.

### PBA Increased the GAT-1 Expression and Function in the Heterozygous Condition Likely Through the Wildtype Allele and the Functional Mutant Allele in Some Cases

PBA is a hydrophobic chaperone, and it may prevent the aberrant interaction of the mutant GAT-1 protein with its wildtype binding partners. For the mutant allele alone, PBA increased most mutant GAT-1 levels apart from those in the E16X mutation. This is likely because the GAT-1(E16X) is severely misfolded, with only 15 amino acids left in the protein peptide, and thus could not be rescued. In the heterozygous conditions with the wildtype allele present, PBA increased GABA uptake for all surveyed mutations. The increase of function in the heterozygous condition is likely due to the wildtype allele. In some cases, such as GAT-1(A288V), the mutant allele could also be functional if rescued and present on the cell surface. This is critical, since all SLC6A1 variant-bearing patients identified so far are heterozygous, suggesting that they can potentially benefit from a treatment option like PBA.

Based on the data, it is likely that the effect of PBA is bidirectional (FIG. 4C). For the wildtype allele, it can promote membrane trafficking while facilitating the disposal of those severely impaired proteins such as GAT-1(E16X), thus making the trafficking more effective for the wildtype allele. Because the patients are heterozygous, this will consequently increase the net GABA uptake at least from the wildtype even if the mutant GAT-1 cannot be rescued. This hypothesis of increasing the wildtype GAT-1 in the presence of a nonfunctional mutant allele was supported by the findings in human iPSC derived cells and the heterozygous knockin mice. This suggests that PBA could potentially be beneficial for all patients carrying SLC6A1 mutations because of the presence of the wildtype allele.

### PBA Treatment Reduced Seizures in Variant-Bearing Knockin Mice

Seizures and abnormal EEGs are common among patients with SLC6A1 mutations, and thus can serve as a good biomarker for evaluating the effect of PBA. GAT-1(S295L) protein alone has no GABA uptake function as described here and in previous work. In SLC6A1^{+/S295L} mice, significant loss of GAT-1 was observed in all major brain regions. PBA treatment for 1-week reduced seizures in the SLC6A1^{+/S295L} heterozygous mice, likely via increasing the functional GAT-1. The magnitude of GAT-1 increase is variable in different brain regions. The magnitude of increase of GABA uptake and GAT-1 protein expression is modest. Based on studies in other epilepsy mouse models Gabrg2^{+/-} and Gabrg2^{+/Q390X}, a minor increase of γ2 subunit reduces seizure severity from Dravet syndrome to infrequent absence or seizure free. This is likely true for the PBA treated mice. The seizure burden in SLC6A1^{+/S295L} mice was reduced by ˜76% with PBA treatment alone. This is likely via the increase of functional GAT-1 and possibly other undefined mechanisms.

Deficits in GABA uptake are a common mechanism underlying the heterogeneous clinical phenotypes associated with SLC6A1 variants, and PBA increases GABA uptake activity. This suggests that PBA could be applied to patients with the same molecular defects regardless of clinical phenotype, including various epilepsy syndromes, autism, neurodevelopmental delay, and others. PBA targets at a root level of disease pathophysiology, thus it is likely disease-modifying.

### PBA is a Feasible Dual Therapy for SLC6A1 Mutations and Possibly for Many Other Genetic Disorders

There is no effective treatment for SLC6A1 mutation mediated disorders to date. Valproic acid has been reported to control seizures but could not improve cognition. This suggests that the impaired cognition is likely caused by the deficit of GAT-1 function instead of being a consequence of seizure activity. Although the detailed mechanisms of action for PBA needs more thorough investigation, the enhanced membrane trafficking and increased functional GAT-1 levels by upregulating the wildtype allele are believed to be major mechanisms underlying the GAT-1 function restoration and seizure mitigation.

There are numerous ion channels and transporters associated with epilepsy, autism, and neurodevelopmental delay. Based on the substantial characterizations of the impact of mutations in GABA_{A} receptors, and more recently in GAT-1, impaired trafficking, and ER-associated degradation (ERAD) are common mechanisms for mutations in GABA receptors, transporters, and beyond. This study, in combination with previous work on the patho-mechanisms of GABA receptors and transporter 1 mutations, provides evidence that PBA may be a feasible treatment option and is disease-modifying (FIG. 10G). Considering PBA is already FDA-approved for pediatric use and is orally bioavailable, with gene therapy and antisense oligonucleotides (ASOs) on the horizon, this treatment could open a new door or even bring a cure when used in combination with other treatment options, for many genetic disorders. This could be achieved by boosting the wildtype functional allele via gene therapy and reducing the ER-retained mutant protein and ER stress via PBA. This dual therapy is proposed as an actionable mode of treatment for SLC6A1 variants mediated disorders and others with similar pathophysiology.

These data show promise in preclinical cell and mouse models, and a pilot clinical trial has been prompted by initial findings (clinicaltrials.gov/ct2/show/nct04937062). It is proposed that PBA increases the wildtype protein forward trafficking. Multiple mechanisms of action could be involved, and future studies with combined multidisciplinary approaches from in vitro and in vivo with differentially tagged wildtype and mutant alleles will elucidate the detailed mechanisms and provide answers. Nevertheless, this study first identified that PBA could be a treatment option for seizure disorders and neurodevelopmental disorders mediated by SLC6A1 variants.

### Example 3

### Dual Therapy for SLC6A1 Variant-Mediated Developmental Epileptic Encephalopathy: Gene Restoration Plus 4-Phenylbutyrate

Previous work identified that a partial or complete loss of GABA uptake function is a major pathophysiology for SLC6A1 mutation-mediated neurodevelopmental disorders in a large cohort of patients. 4-phenylbutyrate (PBA) can partially rescue disease phenotype in numerous patient mutation-bearing cell models and knockin mouse models (S295L and A288V). SLC6A1 mutation-mediated neurodevelopmental disorders are likely life-long due to the existence of the mutation. Because PBA treatment requires high dosage and may produce unwanted side effects, there is great potential in developing alternative treatment strategies, such as gene therapy. Further experiments compared these two mutations in cell and mouse models. Combined treatment with PBA and gene therapy may be the most effective, as it could reduce toxicity resulting from high doses of PBA and prevent gene therapy overcorrection.

### Cell Models

In cell models, HEK293T cells were transfected with SLC6A1, SLC6A1(A288V) or SLC6A1(S295L) cDNAs. The live cells were then fluxed and evaluated for GABA uptake with a 3H radiolabeling GABA uptake assay. In order to evaluate gene expression and protein trafficking, total protein from cell lysates were subjected to SDS-PAGE and immunoblotted for GAT-1 and GFP. Gene expression was evaluated at a variety of gene therapy dosages.

### Mouse Models

Current experiments are testing gene therapy in the knockin mouse models SIC6A1^{+/A28V} and SLC6A1^{+/S295L}. The transgenic mouse hSLC6A1^{lg} was generated with a conditional overexpression of humanized SLC6A1 BAC transgene via PiggyBAC mediated integration, global overexpression of which can be activated via breeding with HprtCre mice. In breeding mice overexpressing hSLC6A1 with SLC6A1^{+/A288V} and SLC6A1^{+/S295L} mice, experiments will determine whether hSLC6A1 overexpression can compensate for A288V or S295L mutation-mediated loss of GABA uptake function. Additional experiments will test if hSLC6A1 overexpression in conjunction with PBA treatment can restore GABA uptake and reduce the necessary PBA dosage or the gene dosage of the human SIC6A1, reducing the risk of unwanted PBA side effects and gene therapy overcorrection. hSLC6A1 overexpressing mice are being bred with SLC6A1^{+/S295L} mice and baseline RT-qPCR data is being collected. Next steps include generating RT-qPCR and western blot data in order to quantify hSLC6A1, SLC6A1^{+/A288V} and SLC6A1^{+/S295L} gene expression and protein production, and later behavioral testing.

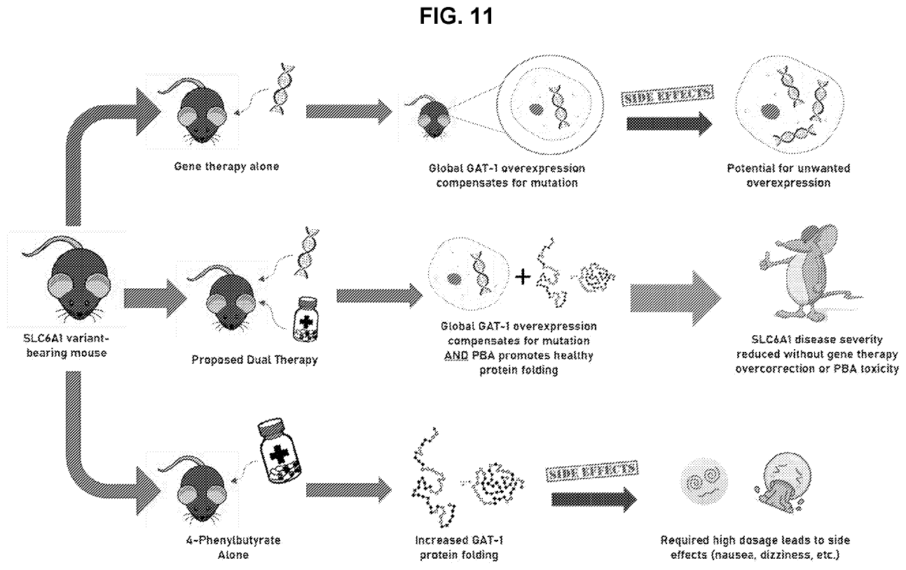

FIG. 11 shows a synopsis of each individual therapy and the combined gene therapy in combination with PBA.

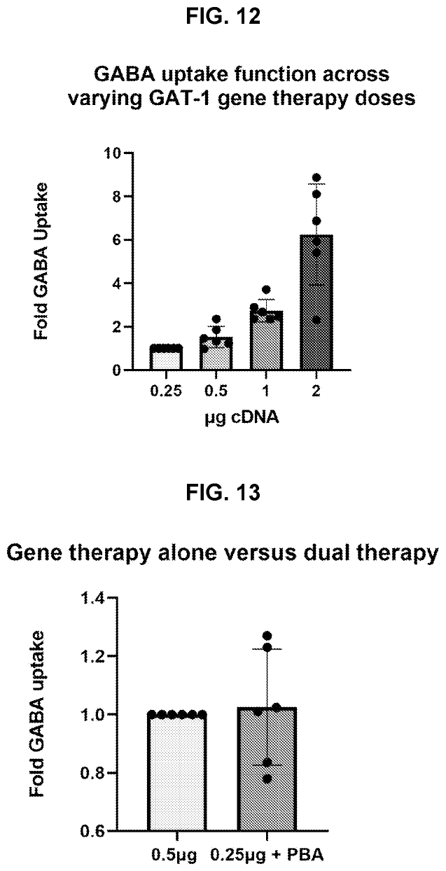

FIG. 12 shows GABA uptake function was assessed in HEK cells transfected with increasing doses of GAT-1 cDNA. 0.25 μg of cDNA was used as a baseline, relative to which the fold GABA uptake from each other treatment group was compared. There were significant (P<0.05) differences in GABA uptake between 0.25 μg and 2 μg, 0.50 μg and 2 μg, and 1 μg vs 2 μg.

FIG. 13 shows GABA uptake function was assessed in HEK cells transfected with either 0.5 μg of GAT-1 cDNA alone or 0.25 μg of GAT-1 cDNA in combination with 2 mM PBA treatment. No significant difference in the amount of GABA uptake was observed between the cDNA alone treatment and the combined cDNA/PBA treatment.

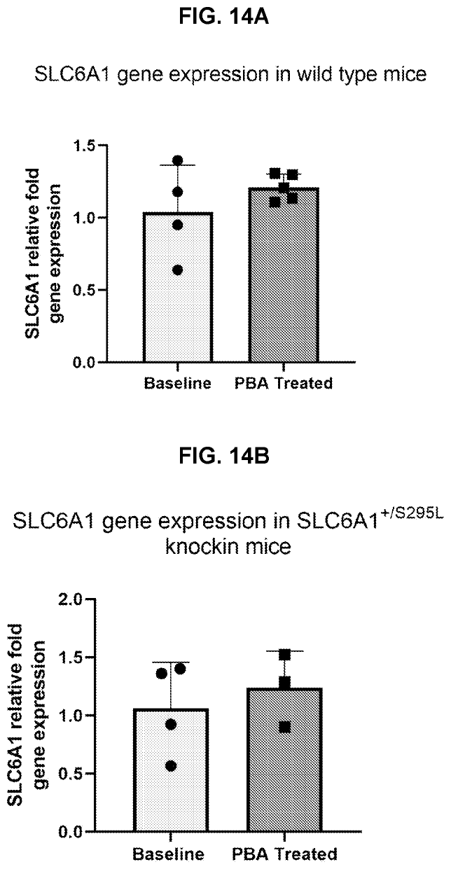

FIGS. 14A-B show RT-qPCR data from a cohort of nine wild-type mice (FIG. 14A) and seven SLC6A1^{+/S295L} mice (FIG. 14B). Five of the wild-type mice and three of the SLC6A1^{+/S295L} mice were treated with PBA for 7 days prior to tissue collection, while the remainder (four wild-type, four SLC6A1^{+/S295L}) were left untreated. There was no significant difference in the amount of SLC6A1 gene expression detected between the treated and untreated mice. Total RNA was extracted from mouse whole-brain tissue.

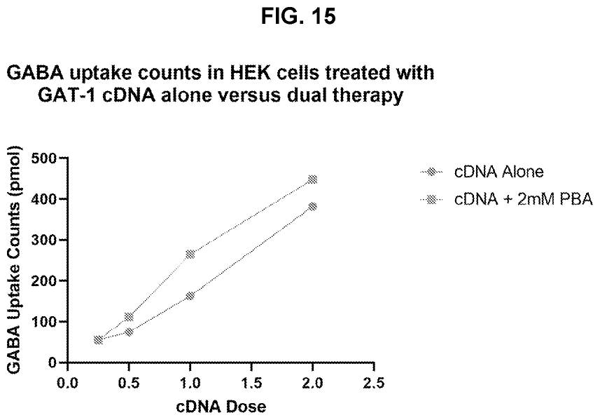

FIG. 15 shows a comparison between GABA uptake function in HEK cells transfected with cDNA alone versus HEK cells transfected with an equal amount of cDNA in conjunction with PBA treatment.

The data highlights the potential utility of a proposed dual therapy. Previous studies transfecting HEK cells with GAT-1 cDNA indicated that higher gene therapy doses result in greater GABA uptake function that to an extent exhibited dose-dependency, and that a treatment approach using a smaller dose of GAT-1 cDNA (0.25 μg) plus PBA lead to similar GABA uptake as was seen with a larger cDNA dose (0.50 μg) alone. Additionally, by comparing cDNA treatment alone versus the proposed dual therapy at a variety of cDNA doses in HEK cells, the dual therapy yielded greater GABA uptake function at all but one dose size.

The ongoing work with GAT-1 mutant knockin mice, though early, has also shown promise. RT-qPCR has shown that PBA treatment does not significantly alter SLC6A1 gene expression.

A dual therapy with gene therapy in combination with PBA could reduce the side effect from PBA and reduce the dosage required for gene therapy. Because the dosage of PBA is easily modifiable, this mode of treatment will help prevent overcorrection from gene therapy.

What is claimed:

1. A method for treating a disease or disorder associated with GABA transporter 1 (GAT-1) dysfunction in a subject, the method comprising:
administering to the subject a therapeutically effective amount of 4-phenylbutyrate, or a pharmaceutically acceptable salt thereof.

2. The method of claim 1, wherein the disease or disorder is associated with one or more solute carrier Family 6 Member 1 (SLC6A1) mutations.

3. The method of claim 2, wherein one or more of the SLC6A1 mutations is a missense or a nonsense mutation.

4. The method of claim 2, wherein one or more of the SLC6A1 mutations is a SLC6A1(A288V) mutation or a SLC6A1(S295L) mutation.

5. The method of claim 1, wherein the method increases GAT-1 function in a plurality of thalamic astrocytes in the subject.

6. The method of claim 1, wherein the method increases GAT-1 function in a plurality of neurons in the subject.

7. The method of claim 1, wherein the disease or disorder is a seizure disorder or a neurodevelopmental disorder.

8. The method of any one of claims 1-7, wherein the disease or disorder is epilepsy, schizophrenia, autism spectrum disorder, attention-deficit/hyperactivity disorder (ADHD), or a learning disorder.

9. The method of claim 1, wherein the method reduces the occurrence of seizures.

10. The method of claim 1, wherein 4-phenylbutyrate, or the pharmaceutically acceptable salt thereof, is administered as a single daily dose.

11. The method of claim 1, wherein 4-phenylbutyrate, or the pharmaceutically acceptable salt thereof, is administered at a dose of 75-125 mg/kg/day.

12. A method for treating a disease or disorder associated with one or more solute carrier Family 6 Member 1 (SLC6A1) mutations in a subject, the method comprising:
administering to the subject a therapeutically effective amount of 4-phenylbutyrate, or a pharmaceutically acceptable salt thereof.

13. The method of claim 12, wherein one or more of the SLC6A1 mutations is a missense or a nonsense mutation.

14. The method of claim 12, wherein one or more of the SLC6A1 mutations is a SLC6A1(A288V) mutation or a SLC6A1(S295L) mutation.

15. The method of claim 12, wherein the disease or disorder is a seizure disorder or a neurodevelopmental disorder.

16. The method of claim 12, wherein the disease or disorder is epilepsy, schizophrenia, autism spectrum disorder, attention-deficit/hyperactivity disorder (ADHD), or a learning disorder.

17. The method of any one of claims 12-16, wherein the method reduces the occurrence of seizures.

18. The method of claim 12, wherein 4-phenylbutyrate, or the pharmaceutically acceptable salt thereof, is administered as a single daily dose.

19. The method of claim 12, wherein 4-phenylbutyrate, or the pharmaceutically acceptable salt thereof, is administered at 75-125 mg/kg/day.

20. A method for treating a disease or disorder associated with GABA transporter 1 (GAT-1) dysfunction in a subject in need thereof, the method comprising:
administering to the subject a therapeutically effective amount of 4-phenylbutyrate, or a pharmaceutically acceptable salt thereof; and
administering a therapeutically effective amount of GAT-1 gene therapy comprising a vector configured to augment wildtype GAT-1 expression in the subject in need thereof.

21. The method of claim 20, wherein the GAT-1 gene therapy comprises expression of wild type SLC6A1.

22. The method of claim 20, wherein the 4-phenylbutyrate and GAT-1 gene therapy function synergistically.

23. The method of claim 20, wherein the therapeutically effective amount of 4-phenylbutyrate is reduced as compared to mono therapy where 4-phenylbutyrate is administered alone.

24. The use of 4-phenylbutyrate, or a pharmaceutically acceptable salt thereof, for the preparation of a medicament for the treatment of a disease or disorder associated with GABA transporter 1 (GAT-1) dysfunction in a subject.

25. The use of claim 24, wherein the disease or disorder is a seizure disorder or a neurodevelopmental disorder.

26. The use of claim 24, wherein the disease or disorder is genetic epilepsy or autism spectrum disorder.

27. The use of claim 24, wherein the medicament reduces the occurrence of seizures.

28. The use of 4-phenylbutyrate, or a pharmaceutically acceptable salt thereof in combination with GAT-1 gene therapy for the preparation of a medicament for the treatment of a disease or disorder associated with GABA transporter 1 (GAT-1) dysfunction in a subject.


SEQUENCE LISTING (4 sequences)

SEQ ID NO: 1 - RNA, 4417 nt, Homo sapiens
  source 1..4417: mol_type = mRNA
gccctcggaagaccgagacagcggagaggttgcgggtgagctgcgctgagcccaggagccgaggagtcgggagcgcagtagcgctgagcccgagcccgagcggccccgcgtcccgagcgcatcggagcggccgagccgcccggatgcagcgcctgtcccgggcagcgcagccccggccgcaggatctcacccagggtggcagaaggaggccttctggagctgacccacccccgacgaccatcagggtgcccttgagccgcaaaactgctgtccacgtggaccgggggtgacatcgcacgtccatctgccaggacccctgcgtccaaattccgagacatggcgaccaacggcagcaaggtggccgacgggcagatctccaccgaggtcagcgaggcccctgtggccaatgacaagcccaaaaccttggtggtcaaggtgcagaagaaggcggcagacctccccgaccgggacacgtggaagggccgcttcgacttcctcatgtcctgtgtgggctatgccatcggcctgggcaacgtctggaggttcccctatctctgcgggaaaaatggtgggggagccttcctgatcccctatttcctgacactcatctttgcgggggtcccactcttcctgctggagtgctccctgggccagtacacctccatcggggggctaggggtatggaagctggctcctatgttcaagggcgtgggccttgcggctgctgtgctatcattctggctgaacatctactacatcgtcatcatctcctgggccatttactacctgtacaactccttcaccacgacactgccgtggaaacagtgcgacaacccctggaacacagaccgctgcttctccaactacagcatggtcaacactaccaacatgaccagcgctgtggtggagttctgggagcgcaacatgcatcagatgacggacgggctggataagccaggtcagatccgctggccactggccatcacgctggccatcgcctggatccttgtgtatttctgtatctggaagggtgttggctggactggaaaggtggtctacttttcagccacatacccctacatcatgctgatcatcctgttcttccgtggagtgacgctgcccggggccaaggagggcatcctcttctacatcacacccaacttccgcaagctgtctgactccgaggtgtggctggatgcggcaacccagatcttcttctcatacgggctgggcctggggtccctgatcgctctcgggagctacaactctttccacaacaatgtctacagggactccatcatcgtctgctgcatcaattcgtgcaccagcatgttcgcaggattcgtcatcttctccatcgtgggcttcatggcccatgtcaccaagaggtccattgctgatgtggcggcctcaggccccgggctggcgttcctggcatacccagaggcggtgacccagctgcctatctccccactctgggccatcctcttcttctccatgctgttgatgctgggcattgacagccagttctgcactgtggagggcttcatcacagccctggtggatgagtaccccaggctcctccgcaaccgcagagagctcttcattgctgctgtctgcatcatctcctacctgatcggtctctctaacatcactcaggggggtatttatgtcttcaaactctttgactactactctgccagtggcatgagcctgctgttcctcgtgttctttgaatgtgtctctatttcctggttttacggtgtcaaccgattctatgacaatatccaagagatggttggatccaggccctgcatctggtggaaactctgctggtctttcttcacaccaatcattgtggcgggcgtgttcattttcagtgctgtgcagatgacgccactcaccatgggaaactatgttttccccaagtggggccagggtgtgggctggctgatggctctgtcttccatggtcctcatccccgggtacatggcctacatgttcctcaccttaaagggctccctgaagcagcgcatccaagtcatggtccagcccagcgaagacatcgttcgcccagagaatggtcctgagcagccccaggcgggcagctccaccagcaaggaggcctacatctagggtgggggccactcaccgacccgacactctcaccccccgacctggctgagtgcgaccaccacttgatgtctgaggataccttccatctcaacctacctcgagtggcgagtccagacaccatcaccacgcagagaggggaggtgggaggacagttagacccctgggtgggccctgccgtgggcaaggatacccggtggcttctggcacctggcgggctggtgacctttttaatccaggccccatcagcatcccacgatcggccttggtaaccgccgcggtagatcatttttatcccgccagggagtgtgatgcaggaagaccacatgcgctcctggcttttaaacctgttcctgactgttctcttactgccgaaacccttgactgttatctcggactttgcaggagttcctttccctccgaacgctgctccatgcacaggaaaagggcattttgtacaatggggacttcccgggaacgcttgctcttaagtaccagaagccggcggagctctggctttcgtgtttttggttttctccttcccaaggcagctggattgaaaaaacaaaacaaaacaaaaaaacccaggggcgtcagtcgatattcccagggccgcttctcctgcagtctgtggagcgtccttgtccccgccgccggaatgaatgagcattctgcagcccgatgtccctgtcccctcctcgccgggccattctgattggacctggcccagtgcaatctgtccagacaagccctgcttgctggaaaactgccacaagcacaattgatctctttttatcgccattccaggggcctcaggtcctactggggaaacttcctataccggagctccagtttctcttaagctgcccaatttcacagagtacaaaatagttgtaggggaaatcaaggtgaaggatctgtccgacagtcaagacggatccacagtaatctttcggtctccttaaactaccaccctcgctgccacccaccccaagctgctgccgcctcaccttccttgaaatttctcagcgggagtctcctcactgccactaaaatccacccagcccactaactgaggagctagtgttaatccagagaaccccccgcaatgtgcttccgagattcagactgcttcattgggaagtatgatttgttcctttctggaattgggctccgtggtggcggcggcacttcaagcaaagacagtttcttgcaagctccagtagctccgcgtgtctcatttgccaggaagatgggttcccacgtagcaaatcgtacattgtgccctgtagctccttagctagttagctcacaagccgtgttttatgactaatccttaataactatggtaaataactgtgactgtggggtttttaatctcttgtcattctcatccaaaagtgaccagcataccagttcttgcaataagatattaccctcagaatattaagcacattattgtagagaaaaaaaaatatgtgtacacatatgaacgcacaacatgcacattcatcctcacatgtggcacgtaaggtctcatttgatattgtgtaggaaatctgaagccttttcctgaggtcatctgtaaaatagtctcattgccaaggcatccccagtgccagctggtgaatccatgatcaaaatgcatacgtattgttaaatgataaggtttagaatgacaggaacccatcactgtgtctcatggtcccacttccccatctgtgtgtgaattcctttagactaagggcaggaagacttccagctttctctttgttcttcaatgtgaaactgagaccaagtctctctaagacaaatgcagtgtatttaatgtttgtaagcaattctaagtgagatgtttggcaagaaatcccctaactgatttccatccaaacctaccttatagagcacaatattaagtgttgtacaattactgtgagaactgtgaatatgtgtaacttttttttagtatttgcccggggggaaaaagatattgtattatcatatatgcttttttgcaataaggatttattctcagaacaccaagtaaatctatctctatataaaaaatatatgtaatatatacatattcaaagtatatacagagcctgttttaaaaaatacagtattatttagtaaaattatctgttctatggaccaaatgtaaaatatttataaatgaagatgcattttaaatgtctataaatggtgtcataactagagcacgggcgttatgtaagtttctaagaatttagaggataaataataaaggttctatgatatacaa

SEQ ID NO: 2 - protein, 599 aa, Homo sapiens [sequence omitted]

SEQ ID NO: 3 - RNA, 4144 nt, Mus musculus
  source 1..4144: mol_type = mRNA
agagagctgagaggttgcaggcgagctgcgcgagcccaggagcccaggcgcccgcgtccaagcgcggccgagcccggagcggcccgcgcagccccggccgcaggctctgtggagaaagcctttaggagaagactcctagcagagatcgattaggctgcaaagctgctgtccacgtggactggagctgacatctcgcgcccacctgccaggatccccgctgccaagtttgtgctccgagacatggcgactgacaacagcaaggtggctgatgggcagatctctactgaggtcagcgaggcccctgtggccagcgacaagcccaaaaccctggtagtcaaggtgcagaagaaggccggggacctccctgaccgggacacatggaagggacgcttcgacttcctcatgtcctgcgtgggctatgccatcggcctgggcaatgtgtggaggttcccttacctctgtgggaaaaacggtggcggggccttcctaatcccatatttcctgacgctcatctttgcgggtgttcctctcttccttttggagtgctccctaggccagtacacctccattgggggcctgggcgtatggaagctggcgcccatgttcaagggtgtgggcctcgcggcagctgtgctgtccttctggctgaacatctactacatcgtcatcatctcctgggccatctactacctgtacaactccttcaccacgaccctgccatggaaacagtgtgacaacccgtggaacactgaccgctgcttctccaactacagcctggtcaataccaccaacatgaccagcgccgtggtggagttctgggagcgcaacatgcaccagatgacagatggactggacaagccaggacagatccgctggcctctggccatcacactggccattgcctgggtgctcgtgtatttctgcatctggaagggtgttggttggactggaaaggtggtctacttctcagccacgtacccctacatcatgcttatcatcctgttcttccgtggagtgacgcttcccggggccaaggaggggatcctcttctacatcacacccaacttccgaaagctgtctgattctgaggtgtggcttgacgccgccacccagatcttcttctcctacgggctgggcctggggtccctgattgctctgggaagctacaactctttccacaacaatgtgtacagggactccatcatcgtttgctgcatcaactcctgcaccagcatgtttgccggattcgtcatcttctccatcgtgggcttcatggctcatgtcaccaagaggtccatagctgatgtggcagcctcaggcccggggctggcattcttggcgtaccctgaggctgtgacacagctacccatctctcccctctgggctatcctcttcttctccatgctgctgatgctgggcattgacagccagttctgtaccgtggagggcttcatcactgccctggtggacgagtaccccagacttctccgcaatcgccgtgaactcttcattgctgccgtgtgcatcgtgtcctacctgattggcctgtctaacatcacccagggtggcatttatgtcttcaaactgtttgattattactctgccagcggcatgagcttgctgttcctggttttcttcgagtgtgtctccatttcctggttttatggtgtcaaccggttctatgacaacatccaggagatggttggctccaggccctgcatctggtggaagctgtgctggtcctttttcacacccatcattgtggcgggcgtgtttctcttcagtgctgtgcagatgacaccactcaccatgggaagctatgttttccccaagtggggccagggcgtgggctggctcatggctctgtcctccatggtgctcatccccgggtacatggcttacatgttcctcaccctgaagggctccctgaagcagcgtctccaggtcatgattcagcccagtgaagatattgtgcgccctgagaatggccctgagcagccgcaggctggcagctcagccagcaaggaggcctacatctaggggtgcagccccccatcacccctacactggcactctggactggctgtacccacaccccttgaagactgaagatactctctgtctccacctacctcaaggggcaggtccagacaccatgaccatgcagagaggggcggtgggggacagtctgaccctgggtgggccctgagtgggcagccgcctctggaggcttccgtagaggccccttagcaggagcaggtggctagcctttgtcactgccactgtagctcctttttatgctgccagagagggtgatagctgcggccacacgctcctggcttttagtctttttttttttttttcagactgttgtcctgtgccccaactatagactgttatccagacttttctgcccgccttggctctgaactggtcatggaccggggcttcggcattttgttcatggccgctgggagccagcaggctctgcctcttccttctcaatttctccttcctgaggcagcttccaggacccacccccagactgttcacagatcagtgcggagagggtcctctgtccctgtgaccctgtgacccgggcagctcagtgtcccctgccctacctccccaggtggcattaacaacaacagcccatccagaagggtcctgtgtactgggaaggaacaaaagaaatcacaagcacaattgccttttttggtcaccatcccaggactttccccaagtgggagctgtggtccttctgagctgcccagtttggcacaaacaatgaccaaggaaatcaaggtgaaggttctaagtcaggaccaatccagctcactctggtttccctcttagactgtcactgccactctgttctggtgtcccccctcacctttctggaaaactttcagatgtacacgcccactcatggagcagggttactccagaggctccgcccaccctaccctggcattaagcttcccttgctagcctgttctagtggggacatcgcatgtcccttctggccttgggttctgtgttggtgaagccagcggagacagtttctggaaagttccagaagctctgtctctctcctctgcggagagggtggactcccatgtagcaaagcgtatgtctcgtcccgtagcttcttagcttgatagctcacaaactgtgtttatgactaatccttaataactatggtgaataactgtgaccgtgggttttttgaatctcttgtcattctcatccagaagtgaccagcacaccagttcttgcaataagctatctccctccctcagagcattaagcacactgtagagaatgcagacacgtatgcacatagaaatgcacacacatgcatacccatcctcacatgtggcatttagcgtcccgtgtgatattgtgtaggcaatctaccagctcttcccgaggccacttgtaacagggttgtgtggctgaggcatctgcctgctcggtggagactctgcgactgaccagcgtgcacagatgcctgtcgacagagtcccaggtgtggaatgcaaggaccctccactgtgtctcgtggcctcaaccccaccccacccccacctgtgtaggaagccctttaggatgagggcaggaggtctccttcttgctgctcggtgttctttgatgtgaaactgagaacaagtctttttgagataaatgcagtgtatttcatgtttgtaagcacctctgagatgtttggcaagaaatcccctgatttccacccaaacttaccttatagagcacaacgttaaaggtcgtacaattactgtgagaactgtgaatatgtgtaactttttttttcagtttttgcccagagggaagaagataattgtattatcatatatgctttcttttttttgcaataaggatttattctcagaacaccaagtaaatttatctctatataaaagtatatgtaatatatgcctattcagactatatacagagcctgttttaaaaaaattacagtattatttagtaaaattatctgttctatggaccaaatgtaaaatatttatacatgaagatgtgttttaaatgtctatcaaatggaatcacagctagaacacgggcgtcatgtacgtttctaagaatttagaggaaaaataataaa

SEQ ID NO: 4 - protein, 599 aa, Mus musculus
MATDNSKVADGQISTEVSEAPVASDKPKTLVVKVQKKAGDLPDRDTWKGRFDFLMSCVGYAIGLGNVWRFPYLCGKNGGGAFLIPYFLTLIFAGVPLFLLECSLGQYTSIGGLGVWKLAPMFKGVGLAAAVLSFWLNIYYIVIISWAIYYLYNSFTTTLPWKQCDNPWNTDRCFSNYSLVNTTNMTSAVVEFWERNMHQMTDGLDKPGQIRWPLAITLAIAWVLVYFCIWKGVGWTGKVVYFSATYPYIMLIILFFRGVTLPGAKEGILFYITPNFRKLSDSEVWLDAATQIFFSYGLGLGSLIALGSYNSFHNNVYRDSIIVCCINSCTSMFAGFVIFSIVGFMAHVTKRSIADVAASGPGLAFLAYPEAVTQLPISPLWAILFFSMLLMLGIDSQFCTVEGFITALVDEYPRLLRNRRELFIAAVCIVSYLIGLSNITQGGIYVFKLFDYYSASGMSLLFLVFFECVSISWFYGVNRFYDNIQEMVGSRPCIWWKLCWSFFTPIIVAGVFLFSAVQMTPLTMGSYVFPKWGQGVGWLMALSSMVLIPGYMAYMFLTLKGSLKQRLQVMIQPSEDIVRPENGPEQPQAGSSASKEAYI